SEQUENCE LISTING (64 sequences)

SEQ ID NO: 1 - DNA, 1629 bp, HUMAN
gcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattatgtaaagctttctgatggaagagagctctgtctggaccccaaggaaaactgggtgcagagggttgtggagaagtttttgaagagggctgagaattcagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagt

SEQ ID NO: 2 - DNA, 3356 bp, HUMAN
gtccaggaactcctcagcagcgcctccttcagctccacagccagacgccctcagacagcaaagcctacccccgcgccgcgccctgcccgccgctgcgatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtgttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactgtcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagtagaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatcttaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataatgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagatta

SEQ ID NO: 3 - DNA, 1750 bp, HUMAN
cctacaggtgaaaagcccagcgacccagtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctttgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagcagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacgggatgaaaccctgtctctactaaaaatacaaaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggt

SEQ ID NO: 4 - DNA, 3939 bp, HUMAN
cctgggtcctctcggcgccagagccgctctccgcatcccaggacagcggtgcggccctcggccggggcgcccactccgcagcagccagcgagccagctgccccgtatgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacaccgccaatcccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactgctacagccttgcccagggtgggccccgtgatgcccactggccaggactggaatcacaccccccagaagacagaccatccatctgccctgctcagagaccccccggagccaggctctcccaggatctcatcactgcgcccccaggccctcagcaacccctccaccctctctgctcagccacagctttccagaagccactcctcgggcagcgtgctgccccttggggagctggagggcaggaggagcaccagggatcggacgagccccgcagagccagaagcagcaccagcaagtgaaggggcagccaggcccctgccccgttttaactccgttcctttgactgacacaggccatgagaggcagtccgagggatcctccagcccgcagctccaggagtctgtcttccacctgctggtgcccagtgtcatcctggtcttgctggctgtcggaggcctcttgttctacaggtggaggcggcggagccatcaagagcctcagagagcggattctcccttggagcaaccagagggcagccccctgactcaggatgacagacaggtggaactgccagtgtagagggaattctaagctggacgcacagaacagtctcttcgtgggaggagacattatggggcgtccaccaccacccctccctggccatcctcctggaatgtggtctgccctccaccagagctcctgcctgccaggactggaccagagcagccaggctggggcccctctgtctcaacccgcagacccttgactgaatgagagaggccagaggatgctccccatgctgccactatttattgtgagccctggaggctcccatgtgcttgaggaaggctggtgagcccggctcaggaccctcttccctcaggggctgcagcctcctctcactcccttccatgccggaacccaggccagggacccaccggcctgtggtttgtgggaaagcagggtgcacgctgaggagtgaaacaaccctgcacccagagggcctgcctggtgccaaggtatcccagcctggacaggcatggacctgtctccagacagaggagcctgaagttcgtggggcgggacagcctcggcctgatttcccgtaaaggtgtgcagcctgagagacgggaagaggaggcctctgcacctgctggtctgcactgacagcctgaagggtctacaccctcggctcacctaagtccctgtgctggttgccaggcccagaggggaggccagccctgccctcaggacctgcctgacctgccagtgatgccaagagggggatcaagcactggcctctgcccctcctccttccagcacctgccagagcttctccagcaggccaagcagaggctcccctcatgaaggaagccattgcactgtgaacactgtacctgcctgctgaacagcctccccccgtccatccatgagccagcatccgtccgtcctccactctccagcctctccccagcctcctgcactgagctggcctcaccagtcgactgagggagcccctcagccctgaccttctcctgacctggcctttgactccccggagtggagtggggtgggagaacctcctgggccgccagccagagccgctctttaggctgtgttcttcgcccaggtttctgcatcttccactttgacattcccaagagggaagggactagtgggagagagcaagggaggggagggcacagacagagagcctacagggcgagctctgactgaagatgggcctttgaaatataggtatgcacctgaggttgggggagggtctgcactcccaaaccccagcgcagtgtcctttccctgctgccgacaggaacctggggctgagcaggttatccctgtcaggagccctggactgggctgcatctcagccccacctgcatggtatccagctcccatccacttctcacccttctttcctcctgaccttggtcagcagtgatgacctccaactctcacccaccccctctaccatcacctctaaccaggcaagccagggtgggagagcaatcaggagagccaggcctcagcttccaatgcctggagggcctccactttgtggccagcctgtggtgctggctctgaggcctaggcaacgagcgacagggctgccagttgcccctgggttcctttgtgctgctgtgtgcctcctctcctgccgccctttgtcctccgctaagagaccctgccctacctggccgctgggccccgtgactttcccttcctgcccaggaaagtgagggtcggctggccccaccttccctgtcctgatgccgacagcttagggaagggcactgaacttgcatatggggcttagccttctagtcacagcctctatatttgatgctagaaaacacatatttttaaatggaagaaaaataaaaaggcattcccccttcatccccctaccttaaacatataatattttaaaggtcaaaaaagcaatccaacccactgcagaagctctttttgagcacttggtggcatcagagcaggaggagccccagagccacctctggtgtcccccaggctacctgctcaggaaccccttctgttctctgagaactcaacagaggacattggctcacgcactgtgagattttgtttttatacttgcaactggtgaattattttttataaagtcatttaaatatctatttaaaagataggaagctgcttatatatttaataataaaagaagtgcacaagctgccgttgacgtagctcgag

SEQ ID NO: 5 - DNA, 1024 bp, HUMAN
atggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgaccagaagggaggaggaagctcactggtggctgttcctgaaggaggccctgcccttataggaacagaagaggaaagagagacacagctgcagaggccacctggattgtgcctaatgtgtttgagcatcgcttaggagaagtcttctatttatttatttattcattagttttgaagattctatgttaatattttaggtgtaaaataattaagggtatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggcttaattattgatgttttcatagagaatataaaaataaagcacttatag

SEQ ID NO: 6 - DNA, 1064 bp, HUMAN
  misc_feature (27)..(27): n = a, c, g, t
cacagccgggtcgcaggcacctccccngccagctctcccgcattctgcacagcttcccgacgcgtctgctgagccccatggcccacgccacgctctccgccgcccccagcaatccccggctcctgcgggtggcgctgctgctcctgctcctggtgggcagccggcgcgcagcaggagcgtccgtggtcactgaactgcgctgccagtgcttgcagacactgcagggaattcacctcaagaacatccaaagtgtgaatgtaaggtcccccggaccccactgcgcccaaaccgaagtcatagccacactcaagaatgggaagaaagcttgtctcaaccccgcatcccccatggttcagaaaatcatcgaaaagatactgaacaaggggagcaccaactgacaggagagaagtaagaagcttatcagcgtatcattgacacttcctgcagggtggtccctgcccttaccagagctgaaaatgaaaaagagaacagcagctttctagggacagctggaaagggacttaatgtgtttgactatttcttacgagggttctacttatttatgtatttatttttgaaagcttgtattttaatattttacatgctgttatttaaagatgtgagtgtgtttcatcaaacatagctcagtcctgattatttaattggaatatgatgggttttaaatgtgtcattaaactaatatttagtgggagaccataatgtgtcagccaccttgataaatgacagggtggggaactggagggtngggggattgaaatgcaagcaattagtggatcactgttagggtaagggaatgtatgtacacatctattttttatacttttttttttaaaaaagaatgtcagttgttatttattcaaattatctcacattatgtgttcaacatttttatgctgaagtttcccttagacattttatgtcttgcttgtagggcataatgccttgtttaatgtccattctgcagcgtttctctttcccttggaaaagagaatttatcattactgttacatttgtacaaatgacatgataataaaagttttatg

SEQ ID NO: 7 - DNA, 1469 bp, HUMAN
agcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaaaaaaaaaaaaaaaacaaaaaaaaaaaa

SEQ ID NO: 8 - DNA, 1256 bp, HUMAN
gcagtagcagcgagcagcagagtccgcacgctccggcgaggggcagaagagcgcgagggagcgcggggcagcagaagcgagagccgagcgcggacccagccaggacccacagccctccccagctgcccaggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacggctccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgcctcttgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccagactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgaggggcccaggcaggcgggcgccaccgccacccgcagcgagggcggagccggccccaggtgctccacatgacagtccctcctctccggagcattttgataccagaagggaaagcttcattctccttgttgttggttgttttttcctttgctctttcccccttccatctctgacttaagcaaaagaaaaagattacccaaaaactgtctttaaaagagagagagag

SEQ ID NO: 9 - DNA, 2121 bp, HUMAN
ctgctcgcggccgccaccgccgggccccggccgtccctggctcccctcctgcctcgagaagggcagggcttctcagaggcttggcgggaaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgcttgatcccccaggccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaa

SEQ ID NO: 10 - DNA, 2098 bp, HUMAN
cctgccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgatatacagcaggtgctcaataaacgattcttagtg

SEQ ID NO: 11 - DNA, 1850 bp, HUMAN
ggcccaggctgaagctcagggccctgtctgctctgtggactcaacagtttgtggcaagacaagctcagaactgagaagctgtcaccacagttctggaggctgggaagttcaagatcaaagtgccagcagattcagtgtcatgtgaggacgtgcttcctgcttcatagataagagcttggagctcggcgcacaaccagcaccatctggtcgcgatggtggacacggaaagcccactctgccccctctccccactcgaggccggcgatctagagagcccgttatctgaagagttcctgcaagaaatgggaaacatccaagagatttcgcaatccatcggcgaggatagttctggaagctttggctttacggaataccagtatttaggaagctgtcctggctcagatggctcggtcatcacggacacgctttcaccagcttcgagcccctcctcggtgacttatcctgtggtccccggcagcgtggacgagtctcccagtggagcattgaacatcgaatgtagaatctgcggggacaaggcctcaggctatcattacggagtccacgcgtgtgaaggctgcaagggcttctttcggcgaacgattcgactcaagctggtgtatgacaagtgcgaccgcagctgcaagatccagaaaaagaacagaaacaaatgccagtattgtcgatttcacaagtgcctttctgtcgggatgtcacacaacgcgattcgttttggacgaatgccaagatctgagaaagcaaaactgaaagcagaaattcttacctgtgaacatgacatagaagattctgaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactgagttccttcagatcagccacaccttttccaggagttctgaagctgacagcactacaaaggagacgggggagcagcacgattttgcacaaatatccaccactttaaccttagagcttggacagtctgagctgtaggtaaccggcatattattccatatctttgttttaaccagtacttctaagagcatagaactcaaatgctgggggaggtggctaatctcaggactgggaag

SEQ ID NO: 12 - DNA, 1609 bp, HUMAN
ttcaagtctttttcttttaacggattgatcttttgctagatagagacaaaatatcagtgtgaattacagcaaacccctattccatgctgttatgggtgaaactctgggagattctcctattgacccagaaagcgattccttcactgatacactgtctgcaaacatatcacaagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactag

SEQ ID NO: 13 - DNA, 3301 bp, HUMAN
  misc_feature (2966)..(2973): n = a, c, g, t
gaattctgcggagcctgcgggacggcggcgggttggcccgtaggcagccgggacagtgttgtacagtgttttgggcatgcacgtgatactcacacagtggcttctgctcaccaacagatgaagacagatgcaccaacgagggtctggaatggtctggagtggtctggaaagcagggtcagatacccctggaaaactgaagcccgtggagcaatgatctctacaggactgcttcaaggctgatgggaaccaccctgtagaggtccatctgcgttcagacccagacgatgccagagctatgactgggcctgcaggtgtggcgccgaggggagatcagccatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaacggcggcacccaggcctccctgcagactccaatggggccagcactggaggggcccacccacatgacttttccattgaccagctctcttcctgtctttgttgtctccctctttctcagttcctctttcttttctaattcctgttgctctgtttcttcctttctgtaggtttctctcttcccttctcccttctcccttgccctccctttctctctcctatccccacgtctgtcctcctttcttattctgtgagatgttttgtattatttcaccagcagcatagaacaggacctctgcttttgcacaccttttccccaggagcagaagagagtgggcctgccctctgccccatcattgcacctgcaggcttaggtcctcacttctgtctcctgtcttcagagcaaaagacttgagccatccaaagaaacactaagctctctgggcctgggttccagggaaggctaagcatggcctggactgactgcagccccctatagtcatggggtccctgctgcaaaggacagtggcagaccccggcagtagagccgagatgcctccccaagactgtcattgcccctccgatcgtgaggccacccactgacccaatgatcctctccagcagcacacctcagccccactgacacccagtgtccttccatcttcacactggtttgccaggccaatgttgctgatggcccctccagcacacacacataagcactgaaatcactttacctgcaggcaccatgcacctcccttccctccctgaggcaggtgagaacccagagagaggggcctgcaggtgagcaggcagggctgggccaggtctccggggaggcaggggtcctgcaggtcctggtgggtcagcccagcacctcgcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctatgtgactctgggtggaagtgcccagcccctgcctgacggnnnnnnngatcactctctgctggcaggattcttcccgctccccacctacccagctgatgggggttggggtgcttctttcagccaaggctatgaagggacagctgctgggacccacctccccccttccccggccacatgccgcgtccctgcccccacccgggtctggtgctgaggatacagctcttctcagtgtctgaacaatctccaaaattgaaatgtatatttttgctaggagccccagcttcctgtgtttttaatataaatagtgtacacagactgacgaaactttaaataaatgggaattaaatatttaaaaaaaaaagcggccgcgaattc

SEQ ID NO: 14 - DNA, 3083 bp, HUMAN
aaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggcttttctactgtacaccccatcccagacgaagacagtccctggatcacctcctccagtgaaaggagcagcacttcaggaggttacatcttttacaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccatttcaacccaatctcacaccccatgggacgaggtcatcaagcaggaagatcgacaacagggacagctgcagcctcagctcataccagccatccaatgcaaggaaggacaacaccaagcccagaggacagttcctggactgatttcaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgtcaatcgttccttatcaggagaccaagacacattccaccccagtggggggtcctttggagttactgcagttactgttggagattccaactctaatgggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttc

SEQ ID NO: 15 - DNA, 2539 bp, HUMAN
ggagtctcttgctctggttcttgctgttcctgctcctgctcccgccgctccccgtcctgctcgcggacccaggggcgcccacgccagtgaatccctgttgttactatccatgccagcaccagggcatctgtgtccgcttcggccttgaccgctaccagtgtgactgcacccgcacgggctattccggccccaactgcaccatccctggcctgtggacctggctccggaattcactgcggcccagcccctctttcacccacttcctgctcactcacgggcgctggttctgggagtttgtcaatgccaccttcatccgagagatgctcatgcgcctggtactcacagtgcgctccaaccttatccccagtccccccacctacaactcagcacatgactacatcagctgggagtctttctccaacgtgagctattacactcgtattctgccctctgtgcctaaagattgccccacacccatgggaaccaaagggaagaagcagttgccagatgcccagctcctggcccgccgcttcctgctcaggaggaagttcatacctgacccccaaggcaccaacctcatgtttgccttctttgcacaacacttcacccaccagttcttcaaaacttctggcaagatgggtcctggcttcaccaaggccttgggccatggggtagacctcggccacatttatggagacaatctggagcgtcagtatcaactgcggctctttaaggatgggaaactcaagtaccaggtgctggatggagaaatgtacccgccctcggtagaagaggcgcctgtgttgatgcactacccccgaggcatcccgccccagagccagatggctgtgggccaggaggtgtttgggctgcttcctgggctcatgctgtatgccacgctctggctacgtgagcacaaccgtgtgtgtgacctgctgaaggctgagcaccccacctggggcgatgagcagcttttccagacgacccgcctcatcctcataggggagaccatcaagattgtcatcgaggagtacgtgcagcagctgagtggctatttcctgcagctgaaatttgacccagagctgctgttcggtgtccagttccaataccgcaaccgcattgccatggagttcaaccatctctaccactggcaccccctcatgcctgactccttcaaggtgggctcccaggagtacagctacgagcagttcttgttcaacacctccatgttggtggactatggggttgaggccctggtggatgccttctctcgccagattgctggccggatcggtgggggcaggaacatggaccaccacatcctgcatgtggctgtggatgtcatcagggagtctcgggagatgcggctgcagcccttcaatgagtaccgcaagaggtttggcatgaaaccctacacctccttccaggagctcgtaggagagaaggagatggcagcagagttggaggaattgtatggagacattgatgcgttggagttctaccctggactgcttcttgaaaagtgccatccaaactctatctttggggagagtatgatagagattggggctcccttttccctcaagggtctcctagggaatcccatctgttctccggagtactggaagccgagcacatttggcggcgaggtgggctttaacattgtcaagacggccacactgaagaagctggtctgcctcaacaccaagacctgtccctacgtttccttccgtgtgccggatgccagtcaggatgatgggcctgctgtggagcgaccatccacagagctctgaggggcaggaaagcagcattctggaggggagagctttgtgcttgtcattccagagtgctgaggccagggctgatggtcttaaatgctcattttctggtttggcatggtgagtgttggggttgacatttagaactttaagtctcacccattatctggaatattgtgattctgtttattcttccagaatgctgaactccttgttagcccttcagattgttaggagtggttctcatttggtctgccagaatactgggttcttagttgacaacctagaatgtcagatttctggttgatttgtaacacagtcattctaggatgtggagctactgatgaaatctgctagaaagttagggggttcttattttgcattccagaatcttgactttctgattggtgattcaaagtgttgtgttcctggctgatgatccagaacagtggctcgtatcccaaatctgtcagcatctggctgtctagaatgtggatttgattcattttcctgttcagtgagatatcatagagacggagatcctaaggtccaacaagaatgcattccctgaatctgtgcctgcactgagagggcaaggaagtggggtgttcttcttgggacccccactaagaccctggtctgaggatgtagagagaacaggtgggctgtattcacgccattggttggaagctaccagagctctatccccatccaggtcttgactcatggcagctgtttctcatgaagctaataaaattcgc

SEQ ID NO: 16 - DNA, 369 bp, HUMAN
atgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgtctgggctgcagaagcgatcagcgatgccagagagaatatccagagattctttggccatggtgcggaggactcgctggctgatcaggctgccaatgaatggggcaggagtggcaaagaccccaatcacttccgacctgctggcctgcctgagaaatactga

SEQ ID NO: 17 - protein, 67 aa, HUMAN
MTSKLAVALLAAFLISAALCEGAVLPRSAKELRCQCIKTYSKPFHPKFIKELRVIESGPHCANTEIM

SEQ ID NO: 18 - protein, 604 aa, HUMAN
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 19 - protein, 360 aa, HUMAN
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 20 - protein, 554 aa, HUMAN
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTANPIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTATALPRVGPVMPTGQDWNHTPQKTDHPSALLRDPPEPGSPRISSLRPQALSNPSTLSAQPQLSRSHSSGSVLPLGELEGRRSTRDRTSPAEPEAAPASEGAARPLPRFNSVPLTDTGHERQSEGSSSPQLQESVFHLLVPSVILVLLAVGGLLFYRWRRRSHQEPQRADSPLEQPEGSPLTQDDRQVELPV

SEQ ID NO: 21 - protein, 107 aa, HUMAN
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 22 - protein, 106 aa, HUMAN
MAHATLSAAPSNPRLLRVALLLLLLVGSRRAAGASVVTELRCQCLQTLQGIHLKNIQSVNVRSPGPHCAQTEVIATLKNGKKACLNPASPMVQKIIEKILNKGSTN

SEQ ID NO: 23 - protein, 300 aa, HUMAN
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 24 - protein, 295 aa, HUMAN
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDGSIRPEELLQMELLLVNKLKWNLAAMTPHDFIEHFLSKMPEAEENKQIIRKHAQTFVASCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 25 - protein, 439 aa, HUMAN
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 26 - protein, 164 aa, HUMAN
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 27 - protein, 468 aa, HUMAN
MVDTESPLCPLSPLEAGDLESPLSEEFLQEMGNIQEISQSIGEDSSGSFGFTEYQYLGSCPGSDGSVITDTLSPASSPSSVTYPVVPGSVDESPSGALNIECRICGDKASGYHYGVHACEGCKGFFRRTIRLKLVYDKCDRSCKIQKKNRNKCQYCRFHKCLSVGMSHNAIRFGRMPRSEKAKLKAEILTCEHDIEDSETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY

SEQ ID NO: 28 - protein, 505 aa, HUMAN
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 29 - protein, 441 aa, HUMAN
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 30 - protein, 742 aa, HUMAN
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 31 - protein, 489 aa, HUMAN
MLMRLVLTVRSNLIPSPPTYNSAHDYISWESFSNVSYYTRILPSVPKDCPTPMGTKGKKQLPDAQLLARRFLLRRKFIPDPQGTNLMFAFFAQHFTHQFFKTSGKMGPGFTKALGHGVDLGHIYGDNLERQYQLRLFKDGKLKYQVLDGEMYPPSVEEAPVLMHYPRGIPPQSQMAVGQEVFGLLPGLMLYATLWLREHNRVCDLLKAEHPTWGDEQLFQTTRLILIGETIKIVIEEYVQQLSGYFLQLKFDPELLFGVQFQYRNRIAMEFNHLYHWHPLMPDSFKVGSQEYSYEQFLFNTSMLVDYGVEALVDAFSRQIAGRIGGGRNMDHHILHVAVDVIRESREMRLQPFNEYRKRFGMKPYTSFQELVGEKEMAAELEELYGDIDALEFYPGLLLEKCHPNSIFGESMIEIGAPFSLKGLLGNPICSPEYWKPSTFGGEVGFNIVKTATLKKLVCLNTKTCPYVSFRVPDASQDDGPAVERPSTE

SEQ ID NO: 32 - protein, 122 aa, HUMAN
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 33 - DNA, 26 bp, HUMAN
agatattgcacgggagaatatacaaa

SEQ ID NO: 34 - DNA, 27 bp, HUMAN
tcaattcctgaaattaaagttcggata

SEQ ID NO: 35 - DNA, 23 bp, HUMAN
tctgcagagttggaagcactcta

SEQ ID NO: 36 - DNA, 21 bp, HUMAN
gccgaggcttttctaccagaa

SEQ ID NO: 37 - DNA, 20 bp, HUMAN
catggcttgatcagcaagga

SEQ ID NO: 38 - DNA, 21 bp, HUMAN
tggaagtgtgccctgaagaag

SEQ ID NO: 39 - DNA, 21 bp, HUMAN
aagcagcaccagcaagtgaag

SEQ ID NO: 40 - DNA, 21 bp, HUMAN
tcatggcctgtgtcagtcaaa

SEQ ID NO: 41 - DNA, 22 bp, HUMAN
acatgccagccactgtgataga

SEQ ID NO: 42 - DNA, 21 bp, HUMAN
ccctgccttcacaatgatctc

SEQ ID NO: 43 - DNA, 23 bp, HUMAN
ggaattcacctcaagaacatcca

SEQ ID NO: 44 - DNA, 23 bp, HUMAN
agtgtggctatgacttcggtttg

SEQ ID NO: 45 - DNA, 22 bp, HUMAN
cagccacaagcagtccagatta

SEQ ID NO: 46 - DNA, 24 bp, HUMAN
cctgactatcaatcacatcggaat

SEQ ID NO: 47 - DNA, 21 bp, HUMAN
ccaggtgctccacatgacagt

SEQ ID NO: 48 - DNA, 24 bp, HUMAN
aaacaaccaacaacaaggagaatg

SEQ ID NO: 49 - DNA, 21 bp, HUMAN
cgtctccacacatcagcacaa

SEQ ID NO: 50 - DNA, 22 bp, HUMAN
tcttggcagcaggatagtcctt

SEQ ID NO: 51 - DNA, 22 bp, HUMAN
gcagaccagcatgacagatttc

SEQ ID NO: 52 - DNA, 20 bp, HUMAN
gcggattagggcttcctctt

SEQ ID NO: 53 - DNA, 23 bp, HUMAN
tgaagttcaatgcactggaactg

SEQ ID NO: 54 - DNA, 20 bp, HUMAN
caggacgatctccacagcaa

SEQ ID NO: 55 - DNA, 23 bp, HUMAN
tggagtccacgagatcatttaca

SEQ ID NO: 56 - DNA, 19 bp, HUMAN
agccttggccctcggatat

SEQ ID NO: 57 - DNA, 21 bp, HUMAN
cactgagttcgccaagagcat

SEQ ID NO: 58 - DNA, 23 bp, HUMAN
cacgccatacttgagaagggtaa

SEQ ID NO: 59 - DNA, 23 bp, HUMAN
gctagtgatcaacagtggcaatg

SEQ ID NO: 60 - DNA, 18 bp, HUMAN
gctggcctctccgttgag

SEQ ID NO: 61 - DNA, 22 bp, HUMAN
tgttcggtgtccagttccaata

SEQ ID NO: 62 - DNA, 22 bp, HUMAN
tgccagtggtagagatggttga

SEQ ID NO: 63 - DNA, 22 bp, HUMAN
gggacatgtggagagcctactc

SEQ ID NO: 64 - DNA, 21 bp, HUMAN
catcatagttcccccgagcat

(19) United States
(12) Patent Application Publication
(10) Pub. No.: US 2009/0112120 A1
(43) Pub. Date: Apr. 30, 2009

(54) DRUG SCREENING AND MOLECULAR DIAGNOSTIC TEST FOR EARLY DETECTION OF COLORECTAL CANCER: REAGENTS, METHODS, AND KITS THEREOF

(75) Inventor: Nancy M. LEE, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: IntelliGeneScan, Inc., Santa Rosa, CA (US)

(21) Appl. No.: 12/180,347

(22) Filed: Jul. 25, 2008

Related U.S. Application Data
(63) Continuation of application No. 11/242,111, filed on Sep. 29, 2005, now abandoned.
(60) Provisional application No. 60/614,746, filed on Sep. 30, 2004.
(60) Provisional application No. 60/651,344, filed on Feb. 8, 2005.

(51) Int. Cl. C12Q 1/68; G01N 33/574; G06F 19/00; C12M 1/00; A61B 10/02

(52) U.S. Cl. 600/572; 435/6; 435/7.23; 702/19; 435/309.1

(57) ABSTRACT

A novel approach to the early detection of colorectal cancer (“CRC”), using a molecular diagnostic test to evaluate grossly normal-appearing colonic tissue for the early detection of colorectal cancer is disclosed. Such grossly normal-appearing colonic mucosal cells may be collected from non-invasive or minimally invasive procedures. The use of novel biomarker panels for drug screening also is disclosed. Such biomaker panels may be used wholly or in part as surrogate endpoints for monitoring effectiveness of a prospective drug in the intervention of pathologies, such as cancers, for example CRC, lung, prostate, and breast, and neurodegenerative diseases, for example Alzheimer's and ALS.

### CLAIM OF PRIORITY

This application claims benefit of U.S. Provisional Application No. 60/614,746, entitled “Molecular Diagnostic Test for Early Detection of Colorectal Cancer: Reagents, Methods, and Kits Thereof,” by Nancy M. Lee et al., filed Sep. 30, 2004 (Attorney Docket No. NLEE-01001US0), and also claims benefit to U.S. Provisional Application No. 60/651,344, entitled “Methods of Use of a Biomarker Panel for Drug Screening,” by Nancy M. Lee et al., filed Feb. 8, 2005 (Attorney Docket No. NLEE-01002US0), each of which is incorporated herein by reference.

### CROSS-REFERENCE TO RELATED APPLICATIONS

This application is related to PCT/US2004/022594, entitled “Biomarker Panel for Colorectal Cancer,” by Nancy M. Lee et al., filed Jul. 14, 2004 (Attorney Docket No. NLEE-01000WO0), which claims priority to U.S. Provisional Application No. 60/488,660, entitled “Molecular Biomarker Panel for Determination of Colorectal Cancer,” by Nancy M. Lee et al., filed Jul. 18, 2003 (Attorney Docket No. CPMC-01000US0), and also to U.S. patent application Ser. No. 10/690,880, entitled “Biomarker Panel for Colorectal Cancer,” by Nancy M. Lee et al., filed Oct. 22, 2003 (Attorney Docket No. CPMC-01000US1), each of which is incorporated herein in full, by reference.

Nucleotide and/or amino acid sequence listings are included in this application in computer-readable form and in hard-copy. The information included in computer-readable form is incorporated herein in full by reference. The information in computer-readable form is also included on diskette, and such information submitted on diskette is incorporated herein in full by reference. Compact diskette No. 1 contains the following file: NLEE01001US1.ST25.txt (created Sep. 29, 2005, 96K). The total number of diskettes submitted is one.

### BACKGROUND

The field of art of this disclosure concerns reagents, methods, and kits for the early detection of colorectal cancer (“CRC”), and methods for drug screening effective in the treatment of pathologies, such as cancers, for example, CRC, lung, prostate, and breast, and neurodegenerative diseases, for example Alzheimer's and ALS. These reagents, methods, and kits are based on a panel of biomarkers that are useful for risk assessment, early detection, establishing prognosis, evaluation of intervention, recurrence of CRC and other such pathologies, and drug discovery for therapeutic intervention.

In the field of medicine, clinical procedures providing for the risk assessment and early detection of CRC have been long sought. Currently, CRC is the second leading cause of cancer-related deaths in the Western world. One picture that has clearly emerged through decades of research into CRC is that early detection is critical to enhanced survival rates.

Thus, one long-sought approach for the early detection of CRC has been the search for biomarkers that are effective in the early detection of CRC, and therefore that are effective for the treatment of CRC. For more than four decades, since the discovery of carcinogenic embryonic antigen (“CEA”), the search for biomarkers effective for early detection of CRC has continued. It is further advantageous for sampling methods used in conjunction with an early diagnostic test for CRC to be minimally invasive or non-invasive. Non-invasive and minimally invasive sampling methods increase patient compliance, and generally reduce cost. Additionally, bioinformatic methods for analysis of complex, multivariate data typical of bioanalysis, yielding a reliable diagnostic evaluation based on such data sets, are also desirable.

Therapeutic intervention for numerous types of cancers, such as CRC, lung, prostate, and breast, includes surgery, chemotherapy, and radiation treatment, and combinations thereof. For CRC, a current area of continued research and development, in addition to search for non-invasive methods for early detection, is in the area of drug development.

One picture that has clearly emerged through decades of research into CRC is that early detection, coupled with effective therapeutic intervention is critical to enhanced survival rates. To date, the most commonly used drug in the treatment of CRC is 5-fluoruracil (“5FU”), which frequently is administered intravenously, in combination with the folic acid vitamin, leucovorin. A strategy referred to as primary chemotherapy is used when metastasis has occurred, and the cancer has spread to different parts of the body. For CRC, the current strategy for primary chemotherapy is the administration of an oral form of 5FU, capecitabine, in combination with Camptosar, a topoisomerase I inhibitor, or Eloxatin, an organometallic, platinum-containing drug that inhibits DNA synthesis.

Currently, strategies for new drug development for CRC include two areas of research: angiogenesis inhibitors, and signal transduction inhibitors.

Novel biopharmaceutical drugs include both protein- and ribozyme-based therapeutics. Humanized antibody-based therapeutics include examples such as Erbitux and Avastin. Erbitux, a signal transduction inhibitor, is aimed at inhibiting epidermal growth factor receptors (“EGFR”) on the surface of cancerous cells. Avastin, an angiogenesis inhibitor, is aimed at inhibiting vascular endothelial growth factor (“VEGF”), which is known to promote the growth of blood vessels. Additionally, Angiozyme, an example of a ribozyme-based therapeutic, is an angiogenesis inhibitor directed against the expression of the VEGF-R1 receptor. New traditional small molecule-based drugs include examples such as Iressa, based on a quinazoline template, and acting as a signal transduction inhibitor, and SU11248, based on an indolinone template, which acts as an anti-angiogenesis inhibitor.

Still, a number of potential drawbacks and uncertainties remain for these nascent drug therapies for CRC. In addition to typical contraindications such as nausea, vomiting, headache, and diarrhea, other more serious side effects, such as gastrointestinal perforation, elevated or lowered blood pressure, extreme fatigue, and internal bleeding have been observed for many of the promising candidates. Additionally, though many of the drug therapies based on angiogenesis inhibition or signal transduction inhibition appear promising, they are in the very early stages of clinical trials.

Accordingly, a need exists in the art for biomarkers that are effective in the early detection of CRC, coupled with sampling methods that are minimally or non-invasive, and bioinformatic methods, which together produce a robust diagnostic test for the early detection of CRC. A need also exists in the art for drug development, which can provide effective treatment prior to the development of cancer for individuals diagnosed with pathologies, such as cancers, for example CRC, lung, prostate, and breast, and neurodegenerative diseases, for example Alzheimer's and ALS, while minimizing serious side effects.

### BRIEF DESCRIPTION OF FIGURES

FIG. 1 is a table listing an embodiment of sequence listings for a panel of biomarkers of the disclosed invention.

FIG. 2 is a distribution plot of control subjects versus test subjects evaluated using an aspect of the panel of biomarkers of FIG. 1, and an aspect of a bioinformatic evaluation of the disclosed invention.

FIG. 3 shows the distribution of the log (base2) expression values for genes, PPAR-γ, IL-8, SAA 1 and COX-2 and their cut-off points.

FIGS. 4A and 4B show that expression of different genes is altered at different sites of MNCM from individuals with a family history of colon cancer.

FIG. 5 displays a flow diagram of an aspect of the bioinformatic process used for evaluating data.

FIG. 6 is an embodiment of a swab sampling and transport system for the minimally invasive sampling of colonic mucosal cells.

FIG. 7 is a flow chart depicting one aspect of the drug screening disclosure.

FIG. 8 is a flow chart depicting another aspect of the drug screening disclosure.

### DETAILED DESCRIPTION

To date, a greater understanding of the biology of CRC has been gained through the research on adenomatous polyposis coli (“APC”), p53, and Ki-ras genes, as well as the corresponding proteins, and related pathways involved regulation thereof. However, there is a distinct difference between research on a specific gene, its expression, protein product, and regulation, and understanding what genes are critical to include in a panel used for the analysis of CRC that is useful in the management of patient care for the disease. Panels that have been suggested for CRC are comprised of specific point mutations of the APC, p53, and Ki-ras, as well as BAT-26, which is a gene that is a microsatellite instability marker.

For CRC, biomarkers for risk assessment and early detection of CRC long have been sought. The difference between risk assessment and early detection is the degree of certainty regarding acquiring CRC. Biomarkers that are used for risk assessment confer less than 100% certainty of CRC within a time interval, whereas biomarkers used for early detection confer an almost 100% certainty of the onset of the disease within a specified time interval. Risk factors may be used as surrogate end points for individuals not diagnosed with cancer, providing that there is an established relationship between the surrogate end point and a definitive outcome. An example of an established surrogate end point for CRC is the example of adenomatous polyps. What has been established is that the occurrence of adenomatous polyps is a necessary, but not sufficient condition for an individual later to develop CRC. This is demonstrated by the fact that 90% percent of all preinvasive cancerous lesions are adenomatous polyps or precursors, but not all individuals with adenomatous polyps go on later to develop CRC.

Adenomatous polyps have been established as surrogate end points for CRC, and adenomatous polyps are macroscopically identifiable by colonoscopy or sigmoidoscopy. During such invasive procedures, biopsy samples can be taken from polyps or lesions for histological evaluation of the tissue. The molecular diagnostic approach disclosed herein may be used on grossly normal-appearing colonic mucosal cells that are not from a macroscopically identifiable polyp or lesion. However, as further disclosed herein, an invasive procedure need not be used to obtain a patient sample for histological evaluation. A non-invasive or minimally-invasive procedure can be employed to obtain, for example, a blood sample, stool sample, or swab of grossly normal-appearing rectal cells, upon which a molecular diagnostic test can be performed to evaluate the presence or absence of CRC. No previously-described approach for early detection of CRC has disclosed the non-invasive or minimally invasive collection of grossly normal-appearing colonic mucosal cells (biopsy or swab of rectal cells), blood samples, and/or stool samples, followed by a molecular and/or protein expression diagnostic test, which can detect changes in the tissue before any untoward histological changes indicating CRC are manifest.

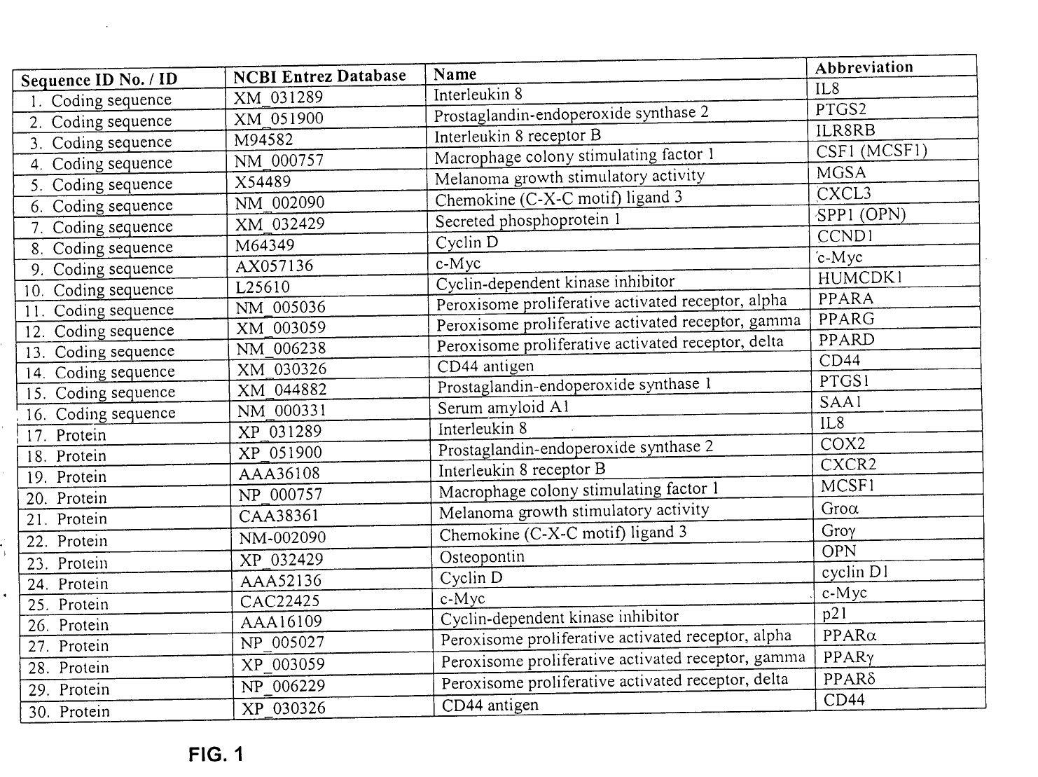

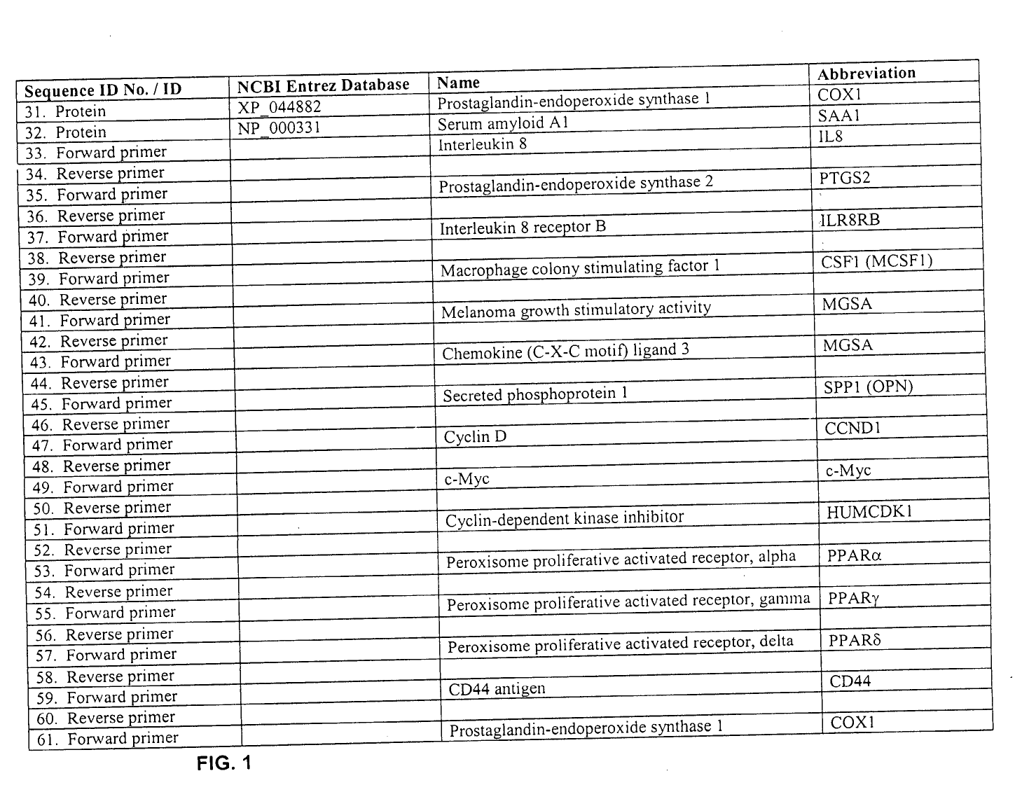

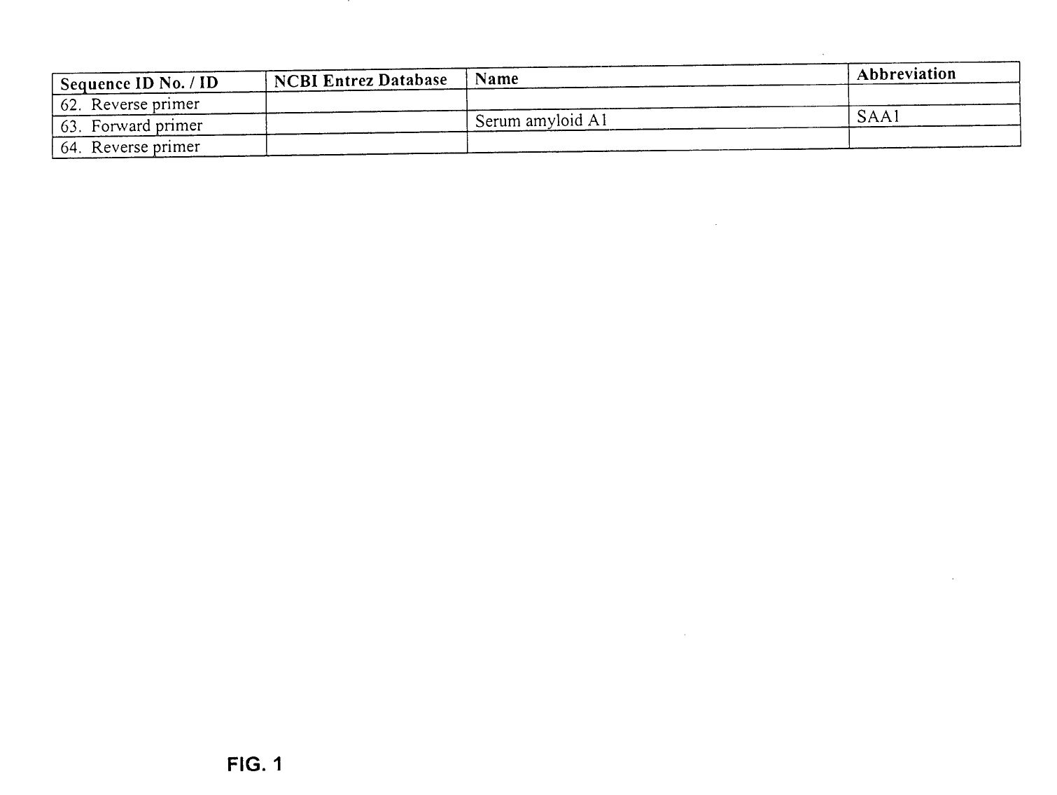

FIG. 1 is a table that gives an overview of the sequence listings included with this disclosure. The table of FIG. 1 lists a panel of biomarkers useful in practicing the disclosed invention. One embodiment of a biomarker panel is the 16 identified coding sequences given by SEQ. ID NOs 1-16, while another embodiment of a biomarker panel is the 16 identified proteins given by SEQ. ID NOs 17-32. These two embodiments represent molecular marker panels that provide the selectivity and sensitivity necessary for the early detection of CRC. It is to be understood that fragments and variants of the biomarkers described in the sequence listings are also useful biomarkers in embodiments of panels used for the early detection of CRC. What is meant by fragment is any incomplete or isolated portion of a polynucleotide or polypeptide in the sequence listing. Further, it is recognized that almost daily, new discoveries are announced for gene variants, particularly for those genes under intense study, such as genes implicated in diseases like cancer. Therefore, the sequence listings given are exemplary of what now is reported for a gene, but it is recognized that for the purpose of an analytical methodology, variants of the gene and their fragments also are included.

In FIG. 1, the entries 1-16 in the table are one aspect of a panel of biomarkers, which are polynucleotide coding sequences, and include the name and abbreviation of the gene. Entries 17-32 in FIG. 1 are another embodiment of a panel of biomarkers, which are protein, or polypeptide, amino acid sequences that correspond to the coding sequences for entries 1-16. A biomarker, as defined by the National Institutes of Health (“NIH”) is a molecular indicator of a specific biological property; a biochemical feature or facet that can be used to measure the progress of disease or the effects of treatment. A panel of biomarkers is a selection of biomarkers, which taken together can be used to measure the progress of disease or the effects of treatment. Biomarkers may be from a variety of classes of molecules. As previously mentioned, there remains a need for biomarkers for CRC having the selectivity and sensitivity required to be effective for early detection of CRC. Therefore, one embodiment of what is disclosed herein is the selection of an effective set of biomarkers that is differentiating in providing the basis for early detection of CRC.

In one aspect of this disclosure, for the early detection of CRC, expression levels of polynucleotides indicated as SEQ. ID NOs 1-16 are determined from cells in samples taken from patients by non-invasive or minimally invasive methods. The contemplated methods include blood sampling, stool sampling, and rectal cell swabbing or biopsy. Such analysis of polynucleotide expression levels frequently is referred to in the art as gene expression profiling. For gene expression profiling, levels of mRNA in a sample are measured as a leading indicator of a biological state—in this case, as an indicator of CRC. One of the most common methods for analyzing gene expression profiling is to create multiple copies from mRNA in a biological sample (said sample taken from a patient as disclosed above, by non- or minimally-invasive methods) using a process known as reverse transcription. In the process of reverse transcription, the mRNA from the sample is isolated from cells in the biological sample, by methods well-known in the art. The mRNA then is used to create copies of the corresponding DNA sequence from which the mRNA was originally transcribed. In the reverse transcription amplification process, copies of DNA are created without the regulatory regions in the gene (i.e., introns). These multiple copies made from mRNA are therefore referred to as “cDNA,” which stands for complementary, or copy DNA. Entries 33-64 are the sets of primers that can be used in the reverse transcription process for each biomarker gene listed in entries 1-16. All nucleotide and amino acid biomarker sequences identified in SEQ. ID NOs 1-64 are found in a printout attached and included as subject matter of this application, and are found on a diskette also included as part of this application and incorporated herein by reference.

Since the reverse transcription procedure amplifies copies of cDNA proportional to the original level of mRNA in a sample, it has become a standard method that allows the identification and quantification of even low levels of mRNA present in a biological sample. Genes either may be up-regulated or down-regulated in any particular biological state, and hence mRNA levels shift accordingly.

In one aspect of this disclosure, a method for gene expression profiling comprises the quantitative measurement of cDNA levels for at least two of the biomarkers of the panel of biomarkers selected from SEQ. ID NOs. 1-16, in a biological sample taken from a patient by a non- or minimally-invasive procedure, such as blood sampling, stool sampling, rectal cell swabbing, and/or rectal cell biopsy. The tissue taken need not be apparently diseased; in fact, the disclosed invention is contemplated to be useful in evaluating even grossly normal-appearing cells for detection of CRC. Such a method for gene expression profiling requires the use of primers, enzymes, and other reagents for the preparation, detection, and quantifying of cDNAs. The method of creating cDNA from mRNA in a sample is referred to as the reverse transcriptase polymerase chain reaction (“RT-PCR”). The primers listed in SEQ. ID NOs 33-64 are particularly suited for use in gene expression profiling using RT-PCR based on the disclosed biomarkers in the biomarker panel. A series of primers were designed using Primer Express Software (Applied Biosystems, Foster City, Calif.). Specific candidates were chosen, and then tested to verify that only cDNA was amplified, and not contaminated by genomic DNA. The primers listed in SEQ. ID NOs 33-64 were specifically designed, selected, and tested accordingly.

The primers listed in SEQ. ID NOs 33-64 are important in the step subsequent to creating cDNA from isolated cellular RNA, for quantitatively amplifying copies in the real time PCR of gene expression products of interest. Optimal primer sequence, and optimal primer length are key considerations in the design of primers. The optimal primer sequence may impact the specificity and sensitivity of the binding of the primer with the template. A primer length between 18-30 bases is considered an optimal range. Theoretically, 18 bases is the minimal length representing a unique sequence, which would hybridize at only one position in most eukaryotic genomes. The primers listed in SEQ. ID NOs 33-64 range in primer length between 21-27 bases, and were designed and validated to amplify cDNA for the panel of nucleotides selected from SEQ. ID NOs 1-16. The specificity of the primers was demonstrated by a single product on 10% polyacrylamide gel electrophoresis (“PAGE”), and a single dissociation curve of the PCR product.

Once the primer pairs have been designed, and validated for specificity, they may be synthesized in large quantities, and stored for convenient future use. Since the PCR reaction is sensitive to buffer concentration and buffer constituents, primers should be maintained in a suitable diluent that will not interfere in the amplification reaction. One example of a suitable diluent is 10 mM Tris buffer, with or without 1 mM EDTA, depending on the assay sensitivity to EDTA. Alternatively, another example of a suitable diluent for the primers is deionized water that is nuclease-free. The primers may be aliquoted in appropriate containers, such as siliconized tubes, and lyophilized if so desired. The liquid or lyophilized samples are preferably stored at refrigeration temperatures defined as long-term for biological samples, which is between about −20° C. to about −70° C. The concentration of primer in the amplification reaction is typically between 0.1 to 0.5 μM. The typical dilution factor from the stock solution to the final reaction mixture is about 10 times, so that the aliquoted stock solution of the primers is typically between about 1 and 5 μM.

In addition to the specifically designed primers listed in SEQ. ID Nos. 33-64, reagents such as one including a dinucleotide triphosphate mixture having all four dinucleotide triphosphates (e.g., dATP, dGTP, dCTP, and dTTP), one having the reverse transcriptase enzyme, and one having a thermostable DNA polymerase, are required for RT-PCR. Additionally buffers, inhibitors, and activators also are required for the RT-PCR process.

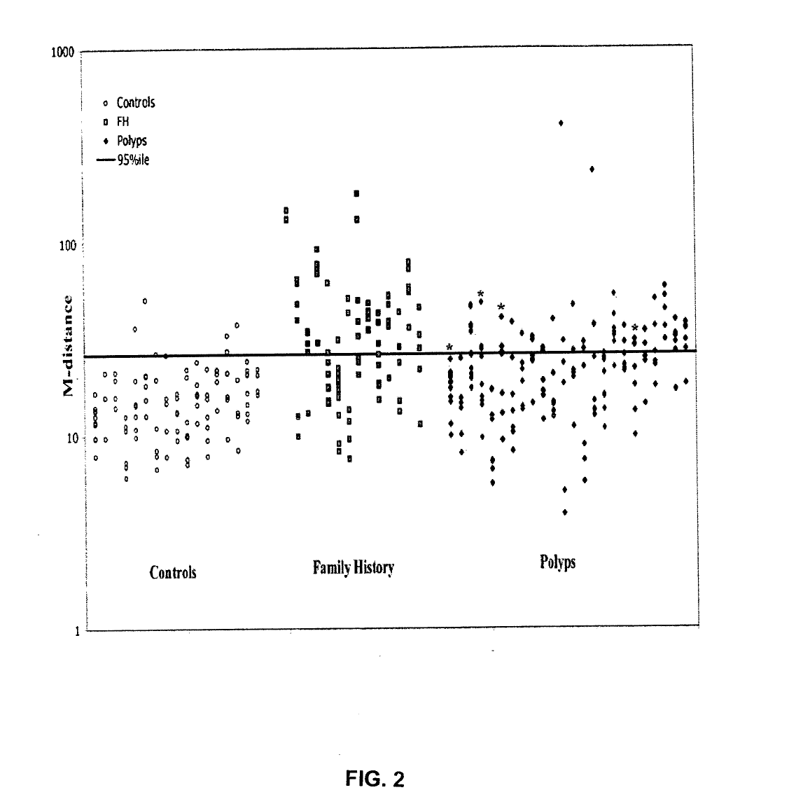

FIG. 2 depicts one aspect of a bioinformatic data reduction process used for the early detection of CRC, showing a distribution of Mahalanobis distance for 17 controls (left), compared with 14 individuals with family history of CRC (middle), and 24 individuals with polyps (right). Tissue samples taken from grossly normal-appearing colonic mucosal tissue were evaluated using the biomarker panel of polynucleotides selected from SEQ. ID NOs. 1-16. The means for the gene expression levels for each of the 16 genes represented by polynucleotides selected from SEQ. ID NOs 1-16 for each control and test subject were calculated in log base 2 domain. The multivariate means, in a 16 dimensional hyperspace, were then determined for the controls, based on a multivariate normal distribution, in order to establish limits of normal expression levels. For each control, the Mahalanobis distance (“M-dist”) from the multivariate mean of the other 16 controls was measured, while the M-dist for each of the test subjects was determined from the multivariate mean of the 17 controls. In each group displayed in FIG. 2, all the biopsies from a single individual form a vertical row. For the individuals with polyps, astericks mark the biopsies from individuals with hyperplastic polyps. The horizontal line indicates the 95th percentile of a chi-square distribution with 16 degrees of freedom. All values above this line (corresponding to an M-dist of about 25) are different from the mean of controls at a level of p<0.05. The data presented clearly show that there is an altered gene expression pattern in grossly normal colonic mucosal tissue samples for the test subjects. The data accordingly demonstrate the enhanced sensitivity and selectivity of a diagnostic test using the biomarker panel of polynucleotides selected from SEQ. ID NOs. 1-16.

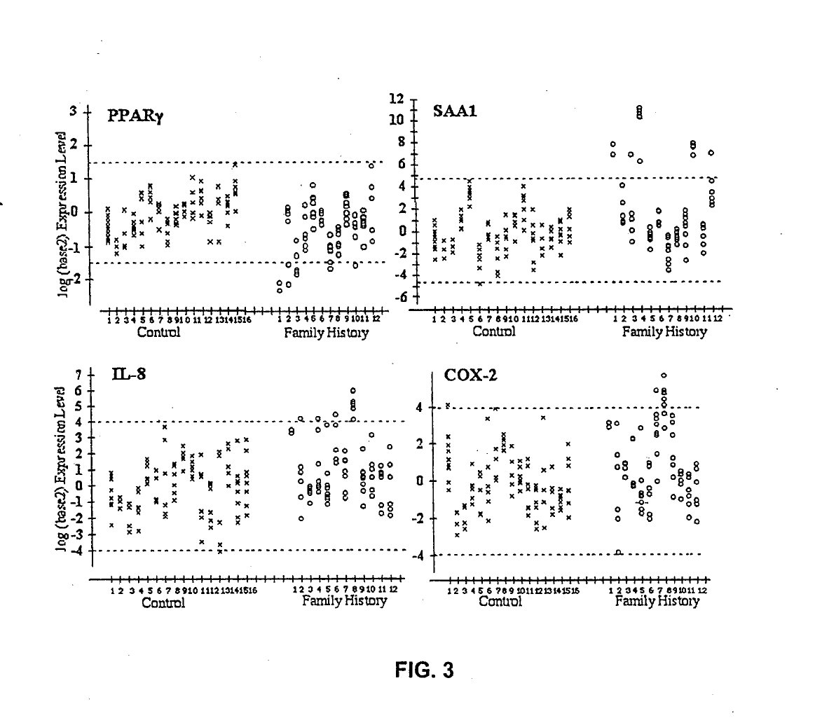

FIG. 3 displays a flow diagram 300 of an aspect of the bioinformatic process used for evaluating the data from samples analyzed using expression profiling of polynucleotides selected from SEQ. ID Nos. 1-16. The goal of the bioinformatic analysis used to analyze the gene expression data for the molecular diagnostic test using the panel of polynucleotides selected from SEQ. ID NOs 1-16 was to use a single, easy-to-calculate measure of abnormality. It is desirable to analyze expression patterns of all genes in the panel selected from SEQ. ID NOs 1-16 by multivariate analysis, since multivariate analysis determines the significance of changes of all expression levels, taken together. There are several kinds of multivariate tests which may be useful for the bioinformatic analysis used to assess the presence or absence of colorectal cancer in patient samples tested using the molecular diagnostic test disclosed herein. Examples of multivariate analysis tests useful in the assessment of data from patient samples tested using the panel of polynucleotide biomarkers selected from SEQ. ID NOs 1-16 include the ANOVA and the Mahalanobis distance (“M-Dist”) tests.

ANOVA is a global test that accounts for correlations among expression levels. It is desirable for the multivariate ANOVA tests to be based on Wilks' lambda criterion and to be carried out on log(base 2) values for the data obtained using the molecular diagnostic test using the panel of polynucleotides selected from SEQ. ID NOs 1-16 to achieve normal distribution of values.

M-dist analysis is another example of a multivariate analysis that summarizes, in a single number, the differences between two patterns of gene expression, taking into account variability of each gene's expression and correlations among pairs of genes. M-dist is often used as a test for outliers (individual cases that are significantly different from all other individual cases in the group) in multivariate data. M-dist can be converted to p-values by reference to a chi-square distribution with degrees of freedom equal to the number of variables (i.e., genes). However, to avoid reliance on an assumption of multivariate normality, it is desirable to compare M-dist for individual cases (i.e., those with polyps) to controls using a rank sum test, the Mann-Whitney test. By using the Mann-Whitney analysis, the inferences concerning differences in expression patterns do not depend on the assumption of multivariate normality. Therefore, this method allows the determination of the significance of all the experimental subjects' expression levels taken together, as well as the significance of each individual expression value.

A working example of the foregoing disclosure is provided below. Hao, C-Y, et al., Alteration of Gene Expression in Macroscopically Normal Colonic Mucosa from Individuals with a Family History of Sporadic Colon Cancer, 11 Clin. Cancer Res., 1400-07 (Feb. 15, 2005). The example presented is provided as a further guide to the practitioner of ordinary skill in the art, and is not to be construed as limiting the invention in any way.

This example was undertaken to investigate whether expression of several genes was altered in morphologically normal colonic mucosa (“MNCM”) of individuals who have not developed colon cancer, but are at high risk of doing so because of a family history of CRC.

Human Subjects

Biopsies of MNCM from the rectum and sigmoid colon were performed at the time of routine colonoscopy from individuals seen at the California Pacific Medical Center (“CPMC”) who had no history of prior colon cancer, and who were free of adenomatous polyps, colon cancer or other colonic lesions at the time of examination. Twelve individuals with a family history of colon cancer in a first-degree relative (Table 3) and sixteen individuals with no known family history of colon cancer were included in the study. Although the information of family cancer history is obtained by patients' self-reports without confirmation from the hospital's cancer registry, a recent study has confirmed the accuracy of self-reported family history with regard to colon cancer. Of the twelve individuals with a family history of colon cancer, two are mother and daughter (cases #6 and 7 in Table 3), two are sister and brother (cases #11 and 12), and the rest are not related. Study subjects ranged in age from 18 to 64 years in the group with a family history of colon cancer, and 16 to 83 years in the control group (the 16-year-old had undergone colonoscopy for chronic abdominal pain). The research protocols for obtaining normal biopsy specimens for study were approved by the CPMC Institutional Review Board. The appropriate procedure for obtaining informed consent was followed for all study subjects.

Extraction and Preparation of RNA and cDNA

Biopsy samples obtained from the segment of colon between the cecum and the hepatic flexure were classified as ascending colon samples; those from the segment of colon between the hepatic flexure and the splenic flexure as transverse colon samples; those from the segment of colon below the splenic flexure as descending colon; those from the winding segment of colon below the descending colon were classified as rectosigmoid colon samples (approximately 5-25 cm from rectum). The number of biopsy samples obtained from each patient varied. Two to eight biopsy samples were obtained from each colon segment, except that only one sample was obtained from the transverse and the descending colon segments in one subject of the family history group. A total of 39 ascending colon, 37 transverse colon, 45 descending colon and 77 rectosigmoid specimens were obtained from the 12 individuals with a family history of colon cancer; and a total of 53 ascending colon, 48 transverse colon, 49 descending colon and 104 rectosigmoid specimens were obtained from the 16 individuals with no family history of colon cancer. All biopsy samples were snap-frozen on dry ice and taken immediately to the laboratory for RNA preparation and reverse transcription as described.

Analysis of Gene Expression

The expression levels of oncogene c-myc, CD44 antigen (“CD44”), cyclooxygenase 1 and 2(“COX-1” and “COX-2”), cyclin D1, cyclin-dependent kinase inhibitor (“p21^{cip/waf1}”), interleukin 8 (“IL-8”), interleukin 8 receptor (“CXCR2”), osteopontin (“OPN”), melanoma growth stimulatory activity (“Groα/MGSA”), GRO3 oncogene (“Groγ”), macrophage colony stimulating factor 1 (“MCSF-1”), peroxisome proliferative activated receptor, alpha, delta and gamma (“PPAR-α, β and γ”) and serum amyloid A 1 (“SAA 1”) were analyzed by quantitative RT-PCR. Quantitative RT-PCR were carried out. In brief, the cycle numbers (“C_{T} value”) were recorded when the accumulated PCR products crossed an arbitrary threshold. To normalize this value, a ΔC_{T} value was determined as the difference between the C_{T} value for each gene tested and the C_{T} value for β-actin. The average ΔC_{T} value for each gene in the control group was calculated. The ΔΔC_{T} value was determined as the difference between the ΔC_{T} value for each individual sample and the average ΔC_{T} value for this gene obtained from the control samples. These ΔΔC_{T} values were then used to calculate relative gene expression values as described. (Applied Biosystems, User Bulletin #2, Dec. 11, 1997). All PCR were performed in duplicate when cDNA samples were available. The results were also verified using histidyl-tRNA synthetase as internal control. Relative gene expression values yielded similar results using either β-actin or his-tRNA synthetase as a reference. Statistical analyses reported here were obtained using β-actin as normalization controls.

Statistical Analysis

Gene expression patterns were compared between individuals with a family history of colon cancer and the control group subjects who had no family history of colon cancer. Rather than testing expression of each gene separately and adjusting for multiple comparisons by methods that reduce statistical power, we tested the expression patterns of all genes by multivariate analysis of variance (“MANOVA”) with Wilks' lambda criterion. This test is a multivariate analog of the F-test for univariate analysis of variance, which tests the equality of means. This type of analysis takes into account correlations among gene expression levels and controls the false-positive rate by providing a single test of whether the expression patterns, based on all the genes in the subset, differ between groups.

If there was evidence that expression patterns differed between groups, we used univariate t-tests to determine which genes were contributing to the global difference. All MANOVA tests were based on the Wilks' lambda criterion and were carried out on log (base 2) of the expression levels, since this transformation was required to achieve normal distributions. Our data consisted of a variable number of samples per subject with different numbers of individuals per group (family history vs. no family history). The analysis included random effects terms for individuals within group and for samples within individuals to account for the sampling scheme. If Yᵢⱼₖ denotes a log 2 gene expression value for the k^{th} sample from the j^{th} patient from the i^{th} group, the statistical model is described mathematically by the equation: Yᵢⱼₖ=M+Aᵢ+Bᵢⱼ+eᵢⱼₖ, where Aᵢ is the (fixed) group effect, Bᵢⱼ is the (random) patient effect, and eᵢⱼₖ is the (random) sample within patient effect.

We also tested whether or not the magnitude of the differential expression (over or under expression) increased along the colon from the ascending portion toward rectum, by defining a variable with value 1 for samples from the ascending, 2 for samples from the transverse, 3 for samples from the descending and 4 for samples from the rectosigmoid portion of the colon. This variable was added to the model so that its effect could be tested for certain genes using univariate ANOVA.

Definition of Cut-Off Point

The log (base 2) of the expression levels of all the biopsy samples from the control group was used to calculate the cut-off point for either up-regulation or down regulation of each gene. A table of tolerance bounds for a normal distribution was used to define cut-off points so that a fraction of the distribution of no more than P would lie above the cut-off point for up-regulated genes or below the cut-off point for down-regulated genes. Each cut-off point was defined by cut-off point=mean+k(SD), where the mean and SD (Standard Deviation) are based on values from the control group. Values of k are found in the table and depend on the P value and the number of normal samples. Owen, D. B., Noncentral t and tolerance limits, in Brimbauim Z W, ed. Handbook of Statistical Tables, Reading, Mass.: Addison-Wesley, 1962, 108-127. Assuming a Gaussian distribution of expression levels of each gene, one would expect less than 1% of the biopsies from a normal population to have an expression level exceeding the 99% tolerance limit (p=0.01).

To calculate the probability that the number of observed samples outside the upper 99 percentile was due to chance in each case, we used the binomial distribution method with p=0.01 and n=the number of samples for each case multiplied by the number of genes tested. For example, for case #1 (Table 3) we had 2 samples; both showed abnormal expression for PPAR-γ and SAA1, one of two for PPAR-δ and neither was abnormal for IL-8 and COX-2. Thus, for this case, 5 of 10 tested were beyond the upper 0.01 boundary. The probability that this happened by chance is 2.4×10⁻⁸. The general formula is given by: Pr{x≧k|p,n}=Σᵢ₌ₖ⁵ⁿ(0.01)ⁱ(0.99)⁵ⁿ⁻ⁱ where k is the number beyond the 99 percentile and n is the number of samples (5 is the number of genes tested).

Results

Altered gene expression in the rectosigmoid mucosa of individuals with a family history of colon cancer:

Twelve individuals (ten women and two men) comprised the group with a family history of colon cancer; 16 individuals (nine women and seven men) served as the control group. (Table 1.) We analyzed a total of 92 ascending colon biopsy samples, 85 transverse colon samples, 94 descending colon biopsy samples and 181 rectosigmoid biopsy samples for levels of expression of 16 genes. Expressions of these genes are known to be altered in the late stages of human colon cancers. We have also shown that some of these genes are altered in the MNCM from surgical resections of colon cancer patients.

Continuing to refer to Table 1, results represent analysis of 104 biopsy samples from the 16 individuals without family history and 77 biopsy samples from 12 individuals with family history of colon cancer in a first-degree relative. Samples were analyzed for gene expression as described in Methods. The numbers in the table represent the expression level relative to the average MC_{T} of the control group. If there is no variation among individuals, the normal gene expression level in the control group should equal to 1. Multivariate analysis using the Wilks Lambda criterion was carried out on log 2 expression values of the 16 genes to determine the significance of the difference between the two groups. Genes are listed from smallest to largest P value.

Multivariate analysis of the expression values of all 16 genes indicated a significant difference in the biopsy samples from the rectosigmoid region (p=0.01) between those with and those without a family history of sporadic colon cancer. Gene expression in biopsy samples from the descending, ascending and transverse colon did not vary significantly between these two groups of individuals (p=0.06, 0.22 and 0.52 respectively). Most of the differences in rectosigmoid biopsy samples were contributed by just five of these genes (Table 1): PPAR-γ, SAA1, IL-8, COX-2 and PPAR-δ. Similar to the alterations of gene expression in the MNCM of cancer patients, we found that the expression levels of SAA1, IL-8 and COX-2 were up-regulated and those of PPAR-γ and PPAR-δ were down-regulated in the MNCM of individuals with a family history of sporadic colon cancer.

The mean (±SD) age in the family history group was younger (45±12 years) than that of the control group (56±16 years), presumably because of heightened awareness of the need for early colonoscopy in the group with a family history of colon cancer. In addition, there is a sex difference between these two groups (ten women and two men in the family history group versus nine women and seven men in the control group). However, we found that sex did not affect the level of gene expression (p=0.67). Moreover, there was no correlation between age and the expression levels of SAA1, IL-8, COX2 and PPAR-γ (all p>0.05) except for PPAR-δ0.01). Nevertheless, abnormal expression (down-regulation) of PPAR-δ increases with age. Thus comparison between younger family history group and older controls, would be biased toward finding fewer, rather than more, abnormal expressions in the family history group. In other words, we may underestimate the incidence of altered expression of PPAR-D in the family history group.

TABLE 1
Gene expression levels in normal rectosigmoid biopsy samples from individuals
with family history of colorectal cancer as compared with controls

| | Controls | | Patients with family history | | |
|---|---|---|---|---|---|
| | (n = 104) | | (n = 77) | | |
| Genes | Range | Mean ± (S.D.) | Range | Mean ± (S.D.) | P Values |
| PPAR-γ | 0.44-1.65 | 1.07 ± 0.41 | 0.20-2.59 | 0.79 ± 0.40 | 0.006 |
| SAA1 | 0.17-22 | 2.16 ± 3.67 | 0.33-2343 | 151 ± 452 | 0.02 |
| IL-8 | 0.14-13 | 1.71 ± 1.94 | 6.84-13 | 6.84 ± 2.82 | 0.02 |
| COX-2 | 0.17-18 | 1.82 ± 2.75 | 0.24-30 | 5.11 ± 9.01 | 0.07 |
| PPAR-δ | 0.39-2.66 | 1.11 ± 0.48 | 0.16-2.22 | 0.89 ± 0.46 | 0.07 |
| CD44 | 0.35-4.13 | 1.14 ± 0.64 | 0.11-4.98 | 1.41 ± 0.78 | 0.12 |
| c-Myc | 0.24-3.66 | 1.21 ± 0.75 | 0.26-4.31 | 1.48 ± 0.82 | 0.14 |
| MCSF-1 | 0.38-22 | 1.81 ± 2.59 | 0.20-11 | 2.04 ± 2.19 | 0.21 |
| Gro-α | 0.01-51 | 2.61 ± 5.48 | 0.34-57 | 5.76 ± 11.63 | 0.22 |
| Gro-γ | 0.16-35 | 2.18 ± 4.29 | 0.12-41 | 2.55 ± 5.91 | 0.25 |
| P21 | 0.51-2.15 | 1.10 ± 0.62 | 0.20-7.68 | 0.90 ± 0.32 | 0.27 |
| PPAR-α | 0.31-2.38 | 1.09 ± 0.55 | 0.26-2.21 | 1.00 ± 0.40 | 0.54 |
| CXCR2 | 0.22-13 | 1.45 ± 1.78 | 0.43-4.44 | 1.49 ± 1.55 | 0.55 |
| OPN | 0.19-13 | 1.66 ± 2.05 | 0.15-12 | 1.41 ± 1.92 | 0.73 |
| CyclinD | 0.34-3.48 | 1.28 ± 0.85 | 0.13-3.21 | 1.29 ± 0.79 | 0.81 |
| COX-1 | 0.27-5.97 | 1.21 ± 0.85 | 0.25-2.63 | 1.09 ± 0.51 | 0.87 |

Comparison with Cut-Off Points for “Normal” Gene Expression

Relative gene expression levels in the rectosigmoid samples varied among individuals, much more so in samples obtained from the individuals with a family history of colon cancer than the corresponding values from the controls (Table 1). We therefore use the expression level of each gene in the control group to define the “normal” expression level for each gene by calculating a cut-off point (p=0.01) for each gene. FIG. 3 shows the distribution of the log (base2) expression values for genes, PPAR-γ, IL-8, SAA 1 and COX-2 and their cut-off points. As expected, less than 1% of the biopsy samples from the control group had expression of these genes above or below the cut-off lines (p=0.01, FIG. 3). However, 21%, 12% and 8% of the biopsy samples from the family history group had expression of SAA1, IL-8 and COX-2, respectively, above the cut-off points, and 12% of them had expression of PPAR-γ below the cut-off point (Table 2).

TABLE 2
Number of biopsy samples (N) with gene expression above/below
the cut-off point in normal individuals and individuals
with a family history of colon cancer

| | | Biopsy samples from | |
|---|---|---|---|
| | Biopsy samples from | individuals with Family | |
| | Normal Controls (n = 104) | History (n = 77) | |
| Genes | N (%) | N (%) | |
| PPAR-γ | 0 | 9 | (12%)†‡ |
| SAAI | 0 | 16 | (21%)*‡ |
| IL-8 | 0 | 9 | (12%)*‡ |
| COX-2 | 1 (1%)* | 6 | (8%)*‡ |
| PPAR-δ | 0 | 2 | (3%)† |
| Gro-γ | 1 (1%)* | 2 | (3%)* |
| PPAR-α | 0 | 2 | (3%)† |

| | | |
|---|---|---|
| Gro-α | 0 | 0 |
| MCSF-1 | 1 (1%)* | 0 |
| OPN | 1 (1%)* | 0 |
| P21 | 0 | 0 |
| CD44 | 1 (1%)* | 0 |
| CXCR2 | 1 (1%)* | 0 |
| c-Myc | 0 | 0 |
| CyclinD | 0 | 0 |
| COX-1 | 0 | 0 |

†with gene expression level below the cut-off point
*with gene expression level above the cut-off point
‡number of patients with alterations are listed in Table 3.

We next analyzed each individual in the family history group (Table 3). The number of biopsy samples which exhibited expression levels below (for PPAR-γ and δ) or above (for IL-8, SAA1 and COX-2) the cut-off point (p=0.01) are indicated. Individuals with all the biopsy samples exhibiting expression levels within the normal range are indicated with a (−) sign. All the grandparents with colon cancers in this study are maternal. Ages of the family member when colon cancer was diagnosed are indicated as follows: *** indicates that colon cancer was diagnosed before 50 years of age; ** indicates before 60 years of age; and * indicates after 60 years of age. Ages of the rest of the family members when colon cancer was diagnosed are not available. None of the twelve patients in the family history group reported other types of cancer in the family except that father of the patient for case #10 had lung cancer in the 1970's.

As evidenced in Table 3, for the five most commonly altered genes, nine of the twelve individuals with a family history of colon cancer had at least one biopsy sample with expression levels below or above the cut-off point. Two individuals (cases #1 and 2) had altered expression of three of these genes in apparently normal rectosigmoid mucosa. In contrast, only one of the sixteen individuals in the control group had altered expression of one of these five genes (see Table 2). The cut-off is set so that 1% of expressions could be false positives. However, the numbers of biopsy samples obtained from each individual are different. To make an adjustment for the number of specimens, we also calculated, for each case, the probability that the number of observed samples outside the upper 99 percentile was due to chance. This calculation was based on the binomial distribution. As shown in Table 3, the observed altered gene expression in seven of the twelve individuals of the family history group is unlikely due to chance (p<0.01). In these seven cases, expressions of at least two of the five genes were altered. In addition, among the sixteen genes analyzed, PPAR-γ and SAA1 are the most frequently altered genes that occurred in five of the twelve individuals with a family history of colon cancer (Table 3).

TABLE 3
Summary of Expression of PPAR-γ, IL-8, SAA1, COX-2 and PPAR-δ in Rectosigmoid
Biopsy Samples from Individuals with a Family History of Colon Cancer

| | | | | # of biopsy | | | | | | # of genes | Probability that |
|---|---|---|---|---|---|---|---|---|---|---|---|
| | | Age | Family member | samples | PPAR-γ | SAA1 | IL-8 | COX-2 | PPAR-δ | with altered | changes are due |
| Case | Sex | (years) | with cancer | analyzed | # of samples with altered expression | | | | | expression | to chance |
| 1 | F | 53 | mother*** | 2 | 2 | 2 | — | — | 1 | 3 | <0.001 |
| 2 | F | 53 | mother* | 6 | 2 | — | 1 | — | 1 | 3 | <0.001 |
| 3 | M | 43 | father* | 5 | 3 | 1 | — | — | — | 2 | <0.001 |
| 4 | F | 47 | mother* | 7 | — | 7 | 1 | — | — | 2 | <0.001 |
| 5 | F | 52 | mother | 8 | — | — | — | — | — | 0 | 1 |
| 6 | F | 52 | father and daughter*** | 6 | — | — | 1 | — | — | 1 | 0.26 |
| 7 | F | 18 | grandfather and sister*** | 8 | 2 | — | — | 1 | — | 2 | <0.01 |
| 8 | F | 35 | mother* and grandmother | 8 | — | — | 8 | 6 | — | 2 | <0.001 |
| 9 | F | 46 | father** | 8 | — | — | — | — | — | 0 | 1 |
| 10 | F | 64 | sister* | 6 | — | 1 | — | — | — | 1 | 0.26 |
| 11 | F | 36 | mother and grandfather | 7 | — | — | — | — | — | 0 | 1 |
| 12 | M | 38 | mother and grandfather | 6 | 1 | 6 | — | — | — | 2 | <0.001 |

| | | | | | |
|---|---|---|---|---|---|
| # of individuals with altered qene expression | 5 | 5 | 4 | 2 | 2 |

Expression of different genes are altered at different sites of MNCM from individuals with a family history of colon cancer.

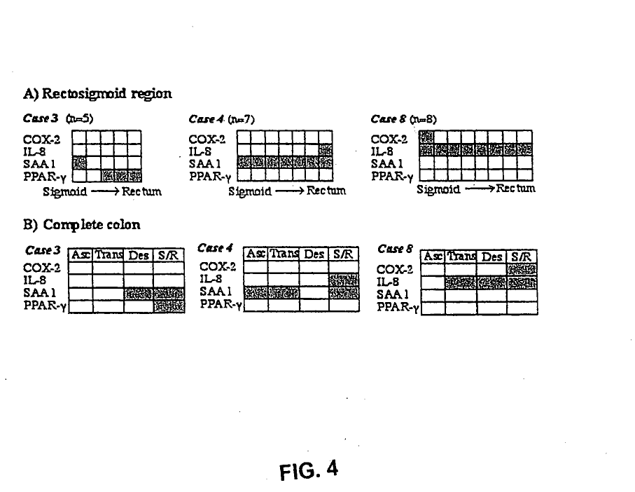

Analysis of individual cases from the family history group showed that different genes were altered in rectosigmoid biopsy samples in different subjects. For instance, SAA1 and PPAR-γ were altered in case #3, IL-8 and SAA1 were altered in case #4; while COX-2 and IL-8 but not SAA1 were altered in case #8 (FIG. 4A). In addition, some genes were altered in all the rectosigmoid biopsy samples from the same patient (such as SAA1 in case #4 and IL-8 in case #8), while others were only altered in some of these biopsy samples (i.e. SAA1 and PPAR-γ in case #3, IL-8 in case #4 and COX-2 in case #8). In addition, some of these alterations are restricted to the rectosigmoid regions, such as IL-8 in case #4; while others can be extended to other regions of the colon, such as SAA1 in case #4 (FIG. 4B).

We also observed that the difference in gene expression between the two groups of individuals increased along the length of the colon for PPAR-γ (p=0.001 for trend) and SAA1 (p<0.001), but not for IL-8 (p=0.20), COX2 (p=0.58), nor PPAR-δ (p=0.54). These results suggest that there is an increasing abnormality along the colon going from the ascending to the rectal portion between the two groups of individuals that can be detected despite reduced numbers of samples toward the ascending portion in this study.

From the foregoing example, it was possible to draw the following conclusions. Approximately 5-10% of colorectal cancers occur among patients with one of the two autosomal dominant hereditary forms of colon cancer (familial adenomatous polyposis and hereditary nonpolyposis colorectal cancer), or who have inflammatory bowel disease (Burt R., Peterson G. M. In: Young G., Rozen, P. & Levin, B. Saunders, ed. in Prevention and Early Detection of Colorectal Cancer, Philadelphia, 171-194 (1996)). Of the remaining colon cancers, approximately 20% are associated with a family history of colon cancer, which is associated with a two-fold increased risk of developing colon cancer (Smith R. A., von Eschenbach A. C., Wender R., et al., American Cancer Society guidelines for the early detection of cancer: update of early detection guidelines for prostate, colorectal, and endometrial cancers, and Update 2001—testing for early lung cancer detection, 51 CA Cancer J. Clin. 38-75; quiz 77-80 (2001)). Although linkage to chromosomes 15q13-14 and 9q22.2-31.2 has been reported in a subset of patients with familial colorectal cancer (Wiesner G. L., Daley D., Lewis S., et al., A subset of familial colorectal neoplasia kindreds linked to chromosome 9g22.2-31.2, 100 Proc Natl Acad Sci USA, 12961-5 (2003)), the genetic basis for most of these cases is not known. In this study, we have demonstrated substantial alterations in the expression of PPAR-γ, IL-8 and SAA1 in the rectosigmoid MNCM from individuals with a family history of sporadic colon cancer, even though these individuals had no detectable colon abnormalities. Our previous study showed that, in addition to PPAR-γ, IL-8 and SAA1, expressions of PPAR-δ, p21, OPN, COX-2, CXCR2, MCSF-1 and CD44 were also altered significantly in the MNCM of colon cancer patients when compared to normal controls without colon cancer, polyps, or family history. These observations suggest that altered expression of genes related to cancer development in the MNCM may be a sequential event and may occur earlier than the appearance of gross morphological abnormalities. For example, altered expression of PPAR-γ, SAA1 and IL-8 may occur in MNCM of individuals who have not developed colon cancer, but are at high risk of doing so; while altered expressions of other genes, such as PPAR-δ, p21, OPN, COX-2, CXCR2, MCSF-1 and CD44, may occur later in MNCM of individuals who have already developed a colon cancer (Chen L-C, Hao C-Y, Chiu Y. S. Y., et al., Alteration of Gene Expression in Normal Appearing Colon Mucosa of APC^{min} Mice and Human Cancer Patients, 64 Cancer Research 3694-3700 (2004)).

Genetic and epigenetic changes have been reported in macroscopically normal tissues for several neoplasms (Tycko B., Genetic and epigenetic mosaicism in cancer precursor tissues, 983 Ann N Y Acad. Sci., 43-54 (2003)). For example, allelic loss has been demonstrated in normal breast terminal ductal lobular units adjacent to primary breast cancers. (Deng G., Lu Y., Ziotnikov G., Thor A. D., Smith H. S., Loss of heterozygosity in normal tissue adjacent to breast carcinomas, 274 Science, 2057-9 (1996)). Such allelic loss is associated with an increased risk of local recurrence (Li Z., Moore D. H., Meng Z. H., Ljung B. M., Gray J. W., Dairkee S. H., Increased risk of local recurrence is associated with allelic loss in normal lobules of breast cancer patients, 62 Cancer Res., 1000-3 (2002)). In addition, normal-appearing colonic mucosal cells from individuals with a prior colon cancer are more resistant to bile acid-induced apoptosis than mucosal cells from individuals with no prior colon cancer (Bernstein C., Bernstein H., Garewal H., et al., A bile acid-induced apoptosis assay for colon cancer risk and associated quality control studies, 59 Cancer Res., 2353-7 (1999); and Bedi A., Pasricha P. J., Akhtar A. J., et al., Inhibition of apoptosis during development of colorectal cancer., 55 Cancer Res., 1811-6 (1995)). Since apoptosis is important in colonic epithelium to eliminate cells with unrepaired DNA damage (Payne C. M., Bernstein H., Bernstein C., Garewal H., Role of apoptosis in biology and pathology: resistance to apoptosis in colon carcinogenesis, 19 Ultrastruct Pathol., 221-48 (1995)), reduction in apoptosis could result in the retention of DNA-damaged cells and increase the risk of carcinogenic mutations.

PPAR-γ is down-regulated in several carcinomas. Ligands of PPAR-γ inhibit cell growth and induce cell differentiation (Kitamura S., Miyazaki Y., Shinomura Y., Kondo S., Kanayama S., Matsuzawa Y., Peroxisome proliferator-activated receptor gamma induces growth arrest and differentiation markers of human colon cancer cells, 90 Jpn J Cancer Res 75-80 (1999)), and loss-of-function mutations in PPAR-γ have been reported in human colon cancer (Sarraf P., Mueller E., Smith W. M., et al., Loss-of-function mutations in PPAR gamma associated with human colon cancer, 3 Mol. Cell, 799-804 (1999)). Thus, our observation of down-regulation in PPAR-γ expression in MNCM may represent an early event that promotes colonic epithelial cell growth and inhibits cellular differentiation. In addition, PPAR-γ also negatively regulates inflammatory response (Welch J. S., Ricote M., Akiyama T. E., Gonzalez F. J., Glass C. K., PPAR gamma and PPAR delta negatively regulate specific subsets of lipopolysaccharide and IFN-gamma target genes in macrophages, 100 Proc Natl Acad Sci USA 6712-7 (2003)). Inflammation favors tumorigenesis by stimulating angiogenesis and cell proliferation (Nakajima N., Kuwayama H., Ito Y., Iwasaki A., Arakawa Y., Helicobacter pylori, neutrophils, interleukins, and gastric epithelial proliferation, 25 Suppl. 1 J Clin Gastroenterol., 98-202 (1997)). Similarly, IL-8 and the acute-phase protein SAA1 modulate the inflammatory process (Dhawan P., Richmond A., Role of CXCL 1 in tumorigenesis of melanoma, 72 J Leukoc Biol., 9-18 (2002); and Urieli-Shoval S., Linke R. P., Matzner Y., Expression and function of serum amyloid A, a major acute-phase protein, in normal and disease states, 7 Curr Opin Hematol., 64-9 (2000)). Up-regulation of pro-inflammatory cytokines and acute phase proteins has been reported in the colon mucosa of individuals with inflammatory bowel disease (Niederau C., Backmerhoff F., Schumacher B., Inflammatory mediators and acute phase proteins in patients with Crohn's disease and ulcerative colitis, 44 Hepatogastroenterology, 90-107 (1997); and Keshavarzian A., Fusunyan R. D., Jacyno M., Winship D., MacDermott R. P., Sanderson I. R., Increased interleukin-8 (IL-8) in rectal dialysate from patients with ulcerative colitis: evidence for a biological role for IL-8 in inflammation of the colon, 94 Am J Gastroenterol., 704-12 (1999)), who are at very high risk of developing colon cancer (Bachwich D. R., Lichtenstein G. R., Traber P. G., Cancer in inflammatory bowel disease, 78 Med Clin North Am., 1399-412 (1994)). Epidemiological observations also suggest that chronic inflammation predisposes to colorectal cancer (Rhodes J. M., Campbell B. J., Inflammation and colorectal cancer: IBD-associated and sporadic cancer compared, 8 Trends Mol Med., 10-6 (2002); and Farrell R. J., Peppercorn M. A., Ulcerative colitis, 359 Lancet 331-40 (2002)). Thus, the observation of down-regulation of PPAR-γ and up-regulation of IL-8 and SAA1 in the normal mucosa of individuals with a family history of sporadic colon cancer and individuals with inflammatory bowel disease may indicate the involvement of common pathways leading to colon carcinogenesis in these two groups.

Our observation of altered expression of genes associated with cancer and inflammation in normal colonic mucosa in some individuals with a family history of colon cancer is consistent with the recent report of association of elevated serum C-reactive protein (“CRP”) concentration prior to the development of colon cancer (Erlinger T. P., Platz E. A., Rifai N., Helzlsouer K. J., C-reactive protein and the risk of incident colorectal cancer., 291 JAMA, 585-90 (2004)). These findings suggest that inflammation is a risk factor for the development of colon cancer in average-risk individuals (id.). However, CRP is a nonspecific marker of inflammation that may indicate inflammation in tissues other than colon. In our study, we have analyzed the tissue where colon cancer arises and would be more specific in assessing the risk of developing colon cancer.

We do not know which cell type is responsible for the observed altered gene expression. There are many cell types in the colonic mucosa, including several types of mucosal epithelial cells, stromal cells and blood-born cells. Studies from our group and others have demonstrated that the up-regulation of COX-2 protein in MNCM is localized primarily to the infiltrating macrophages and secondarily to the epithelial cells in aberrant crypt foci in the MNCM of APC^{min} mice (Chen L-C, Hao C-Y, Chiu Y. S. Y., et al., Alteration of Gene Expression in Normal Appearing Colon Mucosa of APC^{min} Mice and Human Cancer Patients, 64 Cancer Research 3694-3700 (2004); and Hull M. A., Booth J. K., Tisbury A., et al., Cyclooxygenase 2 is up-regulated and localized to macrophages in the intestine of Min mice, 79 Br J Cancer, 1399-405 (1999)). From our previous studies of MNCM of APC^{min} mice, detection of the gene products that are up- or down-regulated in MNCM by immunohistochemical staining was found to be technically difficult, perhaps because the secreted proteins, such as IL-8 and SAA1, are evanescent in tissue sections (Chen L-C, Hao C-Y, Chiu Y. S. Y., et al., Alteration of Gene Expression in Normal Appearing Colon Mucosa of APC^{min} Mice and Human Cancer Patients, 64 Cancer Research 3694-3700 (2004)). Due to the limited amount of the biopsy samples and technical difficulties, we were unable to perform immunohistochemical staining to demonstrate the cell types contributing to the altered gene expression. If the absolute RNA quantities are sufficient, RNA in situ hybridization may be a better method to determine the cellular locations of alterations. Alternatively, laser microdissection followed by RT-PCR may be able to define the cell types involved. Regardless of the cell types responsible for the altered gene expression, our results demonstrate that relative to normal individuals without family history of colon cancer, altered gene expression is present in normal colon mucosa of some individuals with a family history of colon cancer and these individuals are known to have an increased risk of developing colon cancer (Burt R., Peterson G. M. In: Young G., Rozen, P. & Levin, B. Saunders, ed. in Prevention and Early Detection of Colorectal Cancer, Philadelphia, 171-194 (1996)).

Among patients with altered gene expression in the rectosigmoid biopsy samples, some showed alterations in all biopsy samples (i.e., expression of SAA1 in cases #4 and 12), while others showed altered expression in some biopsy samples only (i.e., PPAR-γ in cases #2 and #3, FIG. 2). Since most samples were assayed with multiple genes in duplications to ensure the quality of cDNA, such heterogeneity is unlikely due to technical variation. We speculate that this heterogeneity might reflect the frequency and/or the distribution of “hot spots” in these individuals. It is possible that the individuals with altered gene expression in all rectosigmoid biopsy samples may have wide-spread molecular abnormalities in their rectosigmoid mucosa, while those with altered expression in some of the biopsy samples have discrete hot spots. Thus, individuals in the former group may have a global predisposition to development of colon polyps or cancer, while those in the latter group may have local predisposition. Whether the risks in developing colon cancer or polyps differ between these two groups is unknown. In addition, altered expression of different combination of genes were observed in the rectosigmoid biopsy samples of individuals in the family history group. This observation suggests that different molecular pathways may be involved in the early stages of colon carcinogenesis. Whether altered gene expression in certain molecular pathways is associated with higher risk of polyps or cancer also remains to be determined.

Consistent with the reports of more aberrant crypt foci (the preneoplastic colonic lesions) in the distal colon than in the proximal colon of the sporadic colon cancer patients and the carcinogen-treated mice (Shpitz B., Bomstein Y., Mekori Y., et al., Aberrant crypt foci in human colons: distribution and histomorphologic characteristics, 29 Hum Pathol., 469-75 (1998); and Salim E. I., Wanibuchi H., Morimura K., et al., Induction of tumors in the colon and liver of the immunodeficient (SCID) mouse by 2-amino-3-methylimidazo[4,5-f]quinoline (IQ)-modulation by long chain fatty acids, 23 Carcinogenesis, 1519-29 (2002)), we found that most of the alterations in gene expression were found in the distal colon of the individuals from the family history group. We speculate that the distal colon mucosa of the susceptible individuals may be exposed to higher concentration of exogenous substances present in the stool than mucosa in other colon regions after most of the water is re-absorbed at the end of the large intestine, and such exposure may lead to higher rate of altered gene expression at this region.

We have shown that family history of colon cancer, but not age or sex, is the factor responsible for the observed differences in gene expression in the rectosigmoid mucosa of the two groups. The available information did not indicate any specific difference in diet or medication between these two groups of patients. However, we cannot eliminate the possibility that diet or medication affect gene expression without further study. Not all individuals with a family history of colon cancer will develop cancer or adenomatous polyps of the colon (Smith, R. A., von Eschenbach A. C., Wender, R., et al., American Cancer Society guidelines for the early detection of cancer: update of early detection guidelines for prostate, colorectal, and endometrial cancers, and Update 2001—testing for early lung cancer detection, 51 CA Cancer J. Clin., 38-75; quiz 77-80 (2001).). Consistent with this clinical observation, our analysis also showed that not all the individuals with a family history of colon cancer have altered gene expression in MNCM. Since the genes analyzed in this study are involved in the development of colon cancer, we hypothesize that individuals with altered gene expression in the MNCM may be more susceptible to developing polyps or cancer than those without altered gene expression. To test this hypothesis, a prospective study with a larger number of study subjects will be needed. If such an association is confirmed, it may be possible to identify individuals at increased risk of developing colon cancer by using gene expression analysis of rectosigmoid biopsy samples. Theoretically, it is easier to identify individuals with global alterations in the MNCM than individuals with local alterations by analysis of random MNCM samples. However, if an appropriate panel of genes was selected for analysis using multiple samples, it may have enough predictive power to identify such patients.

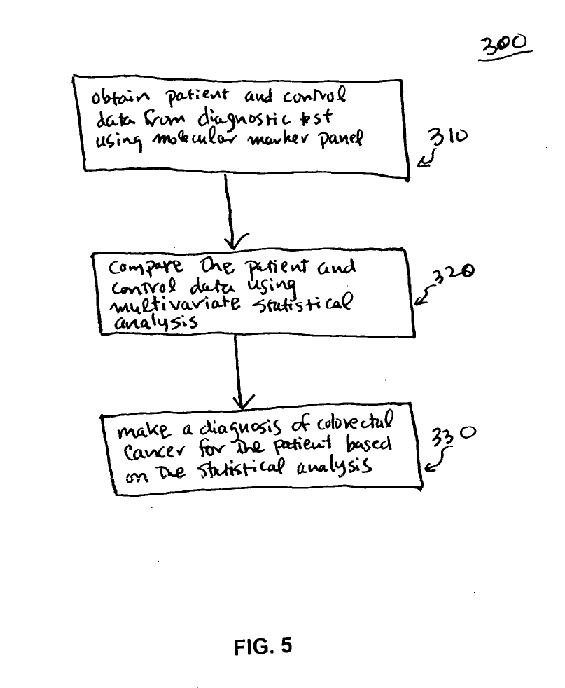

Turning now to FIG. 5, various aspects of FIG. 5 may be implemented using a conventional general purpose or specialized digital computer(s) and/or processor(s) programmed according to the teachings of the present disclosure, as will be apparent to those skilled in the computer arts. Appropriate software coding can be prepared readily by skilled programmers based on the teachings of the present disclosure, as will be apparent to those skilled in the software arts. The invention also may be implemented by the preparation of integrated circuits and/or by interconnecting an appropriate network of component circuits, as will be readily apparent to those skilled in the arts.

Various aspects include a computer program product which is a storage medium having instructions and/or information stored thereon/in which can be used to program a general purpose or specialized computing processor(s)/device(s) to perform any of the features presented herein. The storage medium can include, but is not limited to, one or more of the following: any type of physical media including floppy disks, optical discs, DVDs, CD-ROMs, microdrives, magneto-optical disks, holographic storage devices, ROMs, RAMs, EPROMs, EEPROMs, DRAMs, PRAMS, VRAMs, flash memory devices, magnetic or optical cards, nano-systems (including molecular memory ICs); paper or paper-based media; and any type of media or device suitable for storing instructions and/or information. Various aspects include a computer program product that can be transmitted in whole or in parts and over one or more public and/or private networks wherein the transmission includes instructions and/or information which can be used by one or more processors to perform any of the features presented herein. In various aspects, the transmission may include a plurality of separate transmissions.

Stored on one or more of the computer readable medium (media), the present disclosure includes software for controlling both the hardware of general purpose/specialized computer(s) and/or processor(s), and for enabling the computer(s) and/or processor(s) to interact with a human user or other mechanism utilizing the results of the present invention. Such software may include, but is not limited to, device drivers, operating systems, execution environments/containers, user interfaces and applications.

The execution of code can be direct or indirect. The code can include compiled, interpreted and other types of languages. Unless otherwise limited by claim language, the execution and/or transmission of code and/or code segments for a function can include invocations or calls to other software or devices, local or remote, to do the function. The invocations or calls can include invocations or calls to library modules, device drivers and remote software to do the function. The invocations or calls can include invocations or calls in distributed and client/server systems.

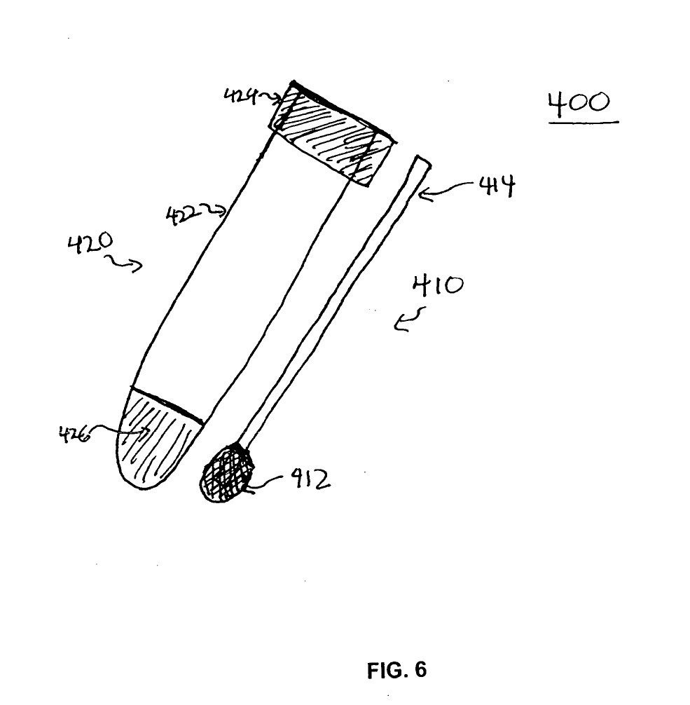

FIG. 6 depicts an aspect of this disclosure having a swab sampling and transport system 400 for the minimally invasive sampling of colonic mucosal cells. The system 400 of FIG. 6 is comprised of a swab 410 and a container 420. A container 420, such as one depicted by the aspect of the disclosure shown in FIG. 6, is configured to stabilize, extract, and store the sample of colonic mucosal cells until the diagnostic test for early detection of CRC using the disclosed biomarker panel can be done on the sample.

The swab 410 has a tip 412 extending from the end of a shaft 414. The tip 410 may be of a number of shapes such as oblate, square, rectangular, round, etc., and has a maximum width of about 0.5 cm to 1.0 cm, and a length of about 1.0 cm to 10.0 cm around the end of the rod. The tip 412 may be composed of a number of materials, such as cotton, rayon, polyester, and polymer foam, for example, or combinations of such materials. The shaft 414 is made of a material with sufficient mechanical strength for effectively swabbing the rectal area, but with enough flexibility to prevent injury. Examples of shaft materials having the strength and flexibility properties for a rectal swab include wood, paper, and a variety of polymeric materials, such as polyester, polystyrene, and polyurethane, and composites of such polymers.

The container 420 has a body 412 and a cap 424. The body 412 may have a variety of lengths and diameters to accommodate a swab 410 having dimensions of the tip 412 and the range of lengths of the shaft 414 as described in the above. The body 412 of the container may be made of a number of polymeric materials, such as polyethylene, polypropylene, polycarbonate, polyfluorocarbon, or glass, while the cap 424 typically is made of a desirable polymeric material, such as the examples given for the body 412. The container 420 has a reagent 426 in the bottom that is suitable for stabilizing and extracting the colonic mucosal cells collected on the swab 410 when swabbing of the rectal area is done as a minimally invasive sampling technique. Additionally, a container 420 having a reagent 426 suitable for stabilizing and extracting a sample of colonic mucosal cells from a stool sample may be used without the need for the swab 410.

The reagent 426 contains a buffered solution of guanidine thiocyanate in a concentration of at least about 0.4M and other tissue denaturing reagents such as a biological surfactant in a concentration of at about between 0.1 to 10%. Desirable biological surfactants can be zwitterionic, such as CHAPS or CHAPSO, non-ionic, such as TWEEN, or any of the alkylglucoside surfactants, or ionic, such as SDS. A variety of buffers, for example, those generally known as Good's buffers, such as Tris, may be used. The concentration of the buffer may vary in order to buffer the reagent 426 effectively to a pH of between about 7.0 to 8.5.

It is further contemplated that the sample taken using an aspect of the disclosure as in FIG. 6 of a swab sampling and transport system 400 can be processed and the data analyzed in a single apparatus using the computer hardware and software disclosed above. That is, the sample obtained from the aspect of the disclosure of FIG. 6 can be analyzed according to FIG. 5 in a single apparatus. However, it is also contemplated that a patient's blood or stool sample can be analyzed in the single apparatus. In one embodiment, one aspect of the apparatus is a first component that is used to carry out RT-PCR for a sample from a patient for gene expression profiling, as described above. Gene expression profiling allows quantifying of cDNA of SEQ. ID Nos 1-16, which is reverse-transcribed from mRNA made by cells in the sample from the patient. The sets of primers from SEQ. ID Nos 33-64 are used in the RT-PCR reaction to prime strands of mRNA corresponding to SEQ. ID Nos 1-16, and thereby to synthesize cDNA corresponding to SEQ. ID Nos 1-16.

After obtaining the cDNAs from the RT-PCR, data are compared by a second component of the apparatus to control data already stored in the apparatus on a storage medium. Multivariate analysis as disclosed above is applied using software to execute instructions for the ANOVA, M-Dist, or other means of multivariate analysis. Based on the statistical analysis, a qualified diagnostician can assess the presence or absence of CRC, the progress of CRC, and/or the effects of treatment of CRC.

In a further aspect of this disclosure, protein expression profiling of patient samples can be carried out for early detection of CRC, using a single apparatus. The term “polypeptide” or “polypeptides” is used interchangeably herein with the term “protein” or “proteins.” As discussed previously, proteins long have been investigated for their potential as biomarkers, with limited success. There is value in protein biomarkers as complementary to polynucleotide biomarkers. Reasons for having the information provided by both types of biomarkers include the current observations that mRNA expression levels are not good predictors of protein expression levels, and that mRNA expression levels tell nothing of the post-translational modifications of proteins that are key to their biological activity. Therefore, in order to understand the expression levels of proteins, and their complete structure, the direct analysis of proteins is desirable.

Disclosed herein are proteins listed in SEQ. ID NOs 17-32, which correspond to the genes indicated in SEQ. ID NOs 1-16. A further aspect of the disclosed invention is to determine expression levels of the proteins indicated by SEQ. ID NOs. 17-32. A sample from the patient, taken by non- or minimally-invasive methods as disclosed above, can be used to prepare fixed cells or a protein extract of cells from the sample. The cells for protein expression profiling can be obtained either through the method of FIG. 6, or alternatively for example by a blood sample or stool sample, or other non-invasive or minimally invasive method (or of course by more conventional invasive methods, including for example sigmoidoscopy and other procedures).

In a first component of the apparatus, the cells or protein extract can be assayed with a panel of antibodies—either monoclonal or polyclonal—against the claimed panel of biomarkers for measuring targeted polypeptide levels. The objective of the assay is to detect and quantify expression of proteins corresponding to the biomarker gene sequences in SEQ. ID NOs 1-16, i.e., SEQ. ID NOs 17-32.

In one aspect of the disclosure contemplated for the method, the antibodies in the antibody panel, which are based on the panel of biomarkers, can be bound to a solid support. The method for protein expression profiling may use a second antibody having specificity to some portion of the bound, targeted polypeptide. Such second antibody may be labeled with molecules useful for detecting and quantifying the bound polypeptides, and therefore in binding to the polypeptide, label it for detection and quantification. Additionally, other reagents are contemplated for labeling the bound polypeptides for detection and quantification. Such reagents may either directly label the bound polypeptide or, analogous to a second antibody, may be a moiety with specificity for the bound polypeptide having labels. Examples of such moieties include but are not limited to small molecules such as cofactors, substrates, complexing agents, and the like, or large molecules such as lectins, peptides, oligonucleotides, and the like. Such moieties may be either naturally occurring or synthetic.

Examples of detection modes contemplated for the disclosed methods include, but are not limited to spectroscopic techniques, such as fluorescence and UV-V is spectroscopy, scintillation counting, and mass spectroscopy. Complementary to these modes of detection, examples of labels for the purpose of detection and quantitation used in these methods include, but are not limited to chromophoric labels, scintillation labels, and mass labels. The expression levels of polynucleotides and polypeptides measured in a second component of the apparatus using these methods may be normalized to a control established for the purpose of the targeted determination. The control data is stored in a computer which is a third component of the apparatus.

A fourth software component compares the data obtained from a patient's or a plurality of patients' samples to the control data. The comparison will comprise at least one multivariate analysis, and can include ANOVA, MANOVA, M-Dist, and others known to those of ordinary skill in the art. Once the statistical analysis and comparison is performed and complete, a physician or other qualified person can make a diagnosis concerning the patient's or patients' CRC status.

Turning now to the drug screening aspect of the present disclosure, it is noted that the panel of biomarkers disclosed herein are genes and expression products thereof that also are known to be involved in the following metabolic pathways and processes: 1) oxidative stress/inflammation; 2) APC/b-catenin pathway; 3) cell cycle/transcription factors; and 4) actions of cytokines and other factors involved in cell/cell communications, growth, repair and response to injury or trauma. There is increasing evidence that these pathways, and hence members of the subject panel of biomarkers, are also involved in many other kinds of cancers than CRC, such as lung, prostate and breast, as well as neurodegenerative diseases, such as Alzheimer's and amyotrophic lateral sclerosis (“ALS”). In such pathologies, genes and expression products thereof involved in these pathways are fundamental to the growth, maintenance and response to stress of cells of many different types. During a pathology such as cancer or neurodegeneration, altered expression of certain altered genes results in a pathological symptom or symptoms, so that a shift in those genes, and expression products thereof are characteristic biomarkers of that particular pathology. In that regard, seemingly unrelated pathologies, such as various cancers and neurodegenerative diseases, are manifestations of very complex pathologies that each involve discrete members of the subject biomarkers, which are genes and expression products thereof drawn from the above group of pathway and processes. As practical evidence of this, it is now appreciated that COX-2 inhibitors have therapeutic value for a wide variety of disorders, including not only colon and other cancers, but for some neurodegenerative diseases as well.

What is disclosed herein is the use of the subject biomarker panel in FIG. 1 in the drug discovery process for pathologies such as cancers, for example CRC, lung prostate, and breast, and neurodegenerative diseases, for example Alzheimer's and ALS. As mentioned in the above, the discrete pattern of altered genes and expression products thereof provides a unique signature for each specific disease, so the panel provides the necessary selectivity for a variety of pathologies. What is meant by drug is any therapeutic agent that is useful in the treatment of a pathology. This includes traditional synthetic molecules, natural products, natural products that are synthetically modified, and biopharmaceutical products, such as polypeptides and polynucleotides, and combinations, extracts and preparations thereof.

Drug screening is part of the first stage of drug development referred to as the drug discovery phase. Prospective drugs that are qualified through the drug screening process are typically referred to as leads, which is to say that in passing the criteria of the screening process they are advanced to further testing in a stage of drug discovery generally referred to as lead optimization. If passing the lead optimization stage of drug discovery, the leads are qualified as candidates, and are advanced beyond the drug discovery stage to the next stage of drug development known as preclinical trials, and are referred to as investigative new drugs (“IND”). If the IND is advanced, it is advanced to clinical trials, where it is tested in human subjects. Finally, if the IND shows promise through the clinical trial stage, after approval from FDA, it may be commercialized. The entire drug development process for a single candidate is known to take 10-15 years and hundreds of millions of dollars in development costs. For that reason, the current strategy within the pharmaceutical drug development community is to focus on the drug discovery stage as effective in weeding out prospective drugs efficiently, and advancing only candidates with high potential for success through the remaining drug development cycle.

In the screening stage of drug discovery, a specific assay for evaluating prospective drugs is performed against a qualified biological model system for which a specific endpoint is monitored. A biomarker panel that is used as a surrogate endpoint for drug screening for pathologies, such as cancers, for example CRC, lung, prostate, and breast, and neurodegenerative diseases, for example Alzheimer's and ALS, is not only a panel useful for early detection of such pathologies, but additionally demonstrates modulation by a drug in a fashion that correlates with a decrease in the pathology occurrence or recurrence. Additionally, one or more members of a biomarker panel useful in the early detection of such pathologies may also be useful as targets for drug screening for such pathologies. As will be discussed subsequently, the biomarkers described by FIG. 1 may be useful both as surrogate endpoints in model biological systems, as well as targets in drug screening.

During the screening phase, large libraries of prospective drugs may be evaluated, representing a throughput of tens of thousands of compounds over a single screening regimen. What is regarded as low-throughput screening (“LTS”) is about 10,000 to about 50,000 prospective drugs, while medium-throughput screening (“MTS”) represents about 50,00 to about 100,00 prospective drugs, and high-throughput screening (“HTS”) is 100,000 to about 500,000 prospective drugs.

What is meant by screening regimen includes both the testing protocol and analytical methodology by which the screening is conducted. The screening regimen, then, includes factors such as the type of biological model that will be used in the test; the conditions under which the testing will be conducted; the type of prospective drug candidates, or library of prospective candidates that will be used; the type of equipment that will be used; and the manner in which the data are collected, processed, and stored. The scale of the screening regimen—LTS, MTS, or HIS—is impacted by factors such as testing protocol (e.g., type of assay), analytical methodology (e.g., miniaturization, automation), and computational capability and capacity. What is meant by biological model system includes whole organism, whole cell, cell lysate, and molecular target. What is meant by prospective drug candidate is any type of molecule, or preparation or suspension of molecules, under consideration for having therapeutic use. For example, the prospective drug candidates could be synthetic molecules, natural products, natural products that are synthetically modified, and biopharmaceutical products, such as polypeptides and polynucleotides, and combinations, extracts, and preparations thereof.

As discussed above, FIG. 1 provides sequence listings of a panel of biomarkers useful in practicing the disclosed invention. One aspect of the disclosure is a biomarker panel of 16 identified coding sequences given in SEQ. ID NOs 1-16, while another aspect of a biomarker panel is the 16 identified proteins given by SEQ. ID NOs 17-31. These two aspects of the present invention provide the selectivity and sensitivity necessary for the early detection of pathologies, such as cancers, for example CRC, lung, prostate, and breast, and neurodegenerative diseases, for example Alzheimer's and ALS.

As previously mentioned, CRC is an exemplary pathology contemplated for development of novel drugs. For CRC, no biomarker or biomarker panel has been identified that has an acceptably high degree of selectivity and sensitivity to be effective for early detection of CRC. Therefore, what is described in FIG. 1 are aspects of biomarker panels that are differentiating in providing the basis for early detection of CRC. Selectivity of a biomarker defined clinically refers to percentage of patients correctly diagnosed. Sensitivity of a biomarker in a clinical context is defined as the probability that the disease is detected at a curable stage. Ideally, biomarkers would have 100% clinical selectivity and 100% clinical sensitivity. To date, no biomarker or biomarker panel has been identified that has an acceptably high degree of selectivity and sensitivity required to be effective for the broad range of needs in patient care management.

The analytical methodology by which the screening is conducted may include the methodologies disclosed above for early detection of CRC, i.e. gene expression profiling from the mRNA of a biological sample to determine the gene expression of biomarkers and how their expression level(s) might have been affected by a prospective drug candidate (including use of RT-PCR), and/or determining protein expression levels of the FIG. 1 polypeptide biomarkers due to application of a prospective drug candidate; and then applying multivariate statistical analysis to determine the statistical significance of the expression levels of the various markers in the panel, with and without the prospective drug candidate(s).

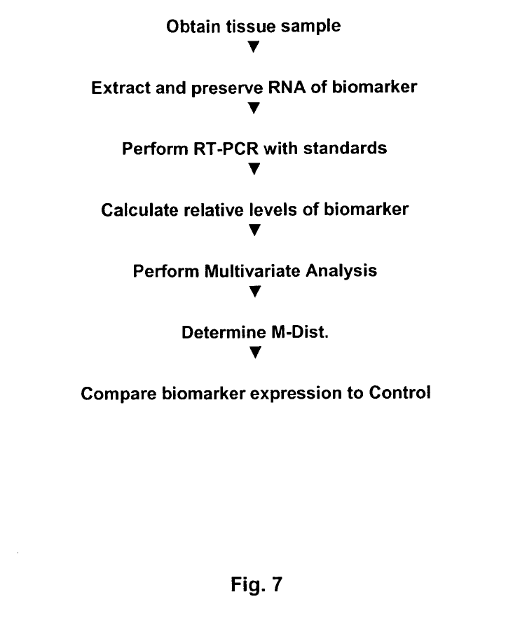

Referring to FIG. 7, one aspect of the drug screening disclosure contemplates obtaining a tissue sample, such as a swab (see FIG. 6), blood sample, or biopsy, which can be taken by, for example, minimally invasive, invasive, or non-invasive means. An appropriate lysis buffer can be used to extract and preserve the RNA of the cells in the tissue sample. RT-PCR then can be carried out on the extracted RNA and converted to cDNA, as disclosed above, using, for example, at least two of the primers listed in SEQ. ID NOs 33-64, specific to the biomarker panel of FIG. 1, to screen the effect of the drug. The results of the assay can then be subjected to a multivariate analysis and M-dist, as disclosed above, and the results compared to control data.

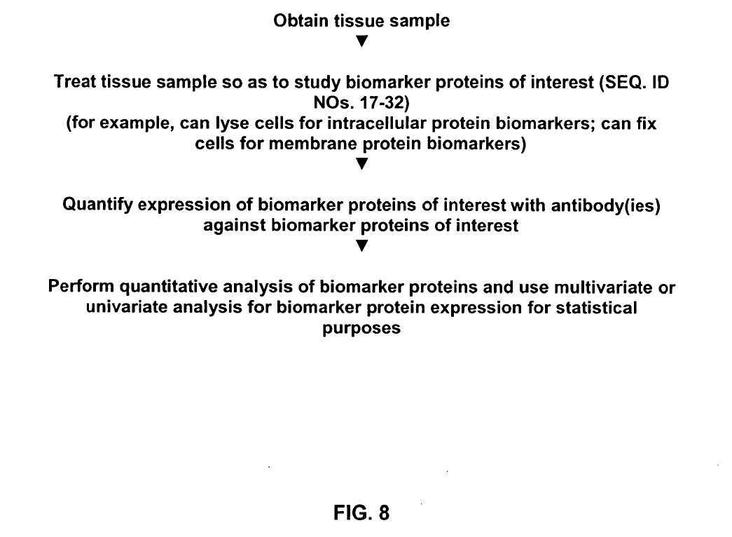

FIG. 8 depicts a further aspect of the drug screening disclosure in which antibodies are made against at least two biomarker proteins listed as SEQ. ID NOs 17-32, and the antibodies are used to assay a biological system, for example whole cells, cell lysates, etc. from, for example, biopsies or other tissue samples as set forth above. The antibodies are used to detect and quantify expression of the biomarker peptides identified by SEQ. ID NOs 17-32, so that the expression of these biomarker peptides can be monitored as a function of dosing the biological system with a potential drug. The results can be subjected to multivariate or univariate analysis and M-dist., as disclosed above, and compared to control data.

What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated.

The references cited above are incorporated by reference in full.

What is claimed:

1. A method for making a reagent composition for the early detection of colorectal cancer, lung cancer, prostate cancer, breast cancer, Alzheimer's and ALS, the method comprising:
synthesizing a pair of primers for each polynucleotide pair from SEQ. ID NOs 33-64;
adjusting to at least one desired concentration in a plurality of separate stock solutions each of said primers, using a diluent;
aliquoting each of said stock solutions of each of said primers into a plurality of containers; and
storing the plurality of containers in long-term storage conditions.

2. The method of claim 1 wherein the method further comprises lyophilizing the aliquoted stock solutions of each of said primer pairs.

3. A method for early detection of colorectal cancer, lung cancer, prostate cancer, breast cancer, Alzheimer's and ALS, the method comprising:
obtaining a tissue sample by a non-invasive or a minimally invasive method from grossly-normal appearing tissue;
isolating RNA from the sample;
amplifying copies of cDNA from the RNA sample using a plurality of pairs of primers selected from the group consisting of SEQ. ID NOs 33-64, to detect a panel of polynucleotides selected from SEQ. ID NOs. 1-16;
quantifying the amplified copies of cDNA; and
using the quantified amplified copies of cDNA to assess at least one of disease progress and treatment effectiveness for at least one of colorectal cancer, lung cancer, prostate cancer, breast cancer, Alzheimer's and ALS.

4. The method as in claim 3 wherein the obtaining step further comprises sampling rectal mucosal cells.

5. The method of claim 3 wherein the obtaining step further comprises one of drawing blood, sampling stool, and taking a rectal biopsy.

6. The method of claim 3 wherein the using step further comprises:
analyzing by multivariate analysis the quantified levels of tissue sample cDNA;
comparing the multivariate analysis of the quantified levels of tissue sample cDNA with a plurality of control data, wherein the comparison determines a significance of differences from the control data to assess the presence of colorectal cancer.

7. The method of claim 6 wherein the analyzing step further comprises using one of an ANOVA test and a Mahalanobis distance test.

8. A method for early detection of colorectal cancer and for evaluation of treatment efficacy of colorectal cancer, the method comprising the steps of:
obtaining by a non-invasive or minimally-invasive method a tissue sample containing cells that grossly appear cancer-free;
generating a plurality of antibodies having different specificities against each of the polypeptides identified by SEQ. ID NOs 17-32;
assaying for expression of polypeptides in a panel of polypeptides identified by SEQ. ID NOs 17-32 with the plurality of antibodies, wherein the assaying step allows for quantifying specific binding of the antibodies to the polypeptides;
quantifying the levels of each of the different polypeptides in the panel of polypeptides based on the quantified specific antibody binding; and
analyzing the quantified levels of each of the different polypeptides in the panel of polypeptides, wherein the quantified levels are used to assess at least one of the presence, progress, and treatment of colorectal cancer.

9. The method of claim 8 wherein the obtaining step further comprises one of sampling blood, sampling stool, swabbing for colonic cells, and performing a rectal biopsy.

10. A method for analyzing data for the early detection and treatment monitoring of colorectal cancer, the method comprising the following steps:
obtaining a plurality of quantified levels of cDNA for polynucleotides selected from SEQ. ID Nos. 1-16 from a patient sample, wherein the sample is taken by a non-invasive method or a minimally-invasive method;
comparing said data from the patient sample to a plurality of stored control data using multivariate statistical analysis; and
making a determination concerning one of diagnosis of colorectal cancer, colorectal cancer progress, and treatment efficacy for the patient based on the comparison.

11. A machine readable medium having instructions stored thereon that, when executed by one or more processors, cause a system to:
obtain the data of quantified levels of cDNA for polynucleotides listed in SEQ. ID NOs. 1-16, wherein the quantified levels of cDNA are from a patient tissue sample and a control tissue sample;
compare the quantified levels of cDNA from the patient tissue sample to the quantified levels of cDNA from the control tissue sample using at least one multivariate statistical analysis; and
provide said multivariate statistical analysis for evaluation by an individual trained to evaluate colorectal cancer.

12. A computer signal embodied in a transmission medium, comprising:
a code segment including instruction for obtaining quantified levels of cDNA for polynucleotides selected from SEQ. ID NOs. 1-16, wherein the quantified levels of cDNA are from a patient tissue sample;
a code segment including instruction for comparing the quantified levels of cDNA from the patient tissue sample to a plurality of control data using multivariate statistical analysis; and
a code segment including instruction for making a diagnosis of colorectal cancer for the patient tissue sample based on the comparison.

13. A computer signal embodied in a transmission medium, comprising:
a code segment including instruction for obtaining quantified levels of polypeptides selected from SEQ. ID NOs. 17-33, wherein the quantified levels of polypeptides are from a patient sample containing colonic mucosal cells;
a code segment including instruction for comparing the quantified levels of polypeptides from the patient sample to a plurality of control data using multivariate statistical analysis; and
a code segment including at least one instruction based on the comparison for at least one of a diagnosis of colorectal cancer, a progress of colorectal cancer, and an efficacy of treatment of colorectal cancer.

14. A kit for use in the early detection of colorectal cancer, the kit comprising:
a collection container for receiving a sample containing rectal mucosal cells obtained through a non-invasive procedure, wherein the collection container is configured to stabilize and store the sample; and
at least one reagent that is used in the analysis of polynucleotide expression levels, wherein the polynucleotides are selected from SEQ. ID Nos. 1-16.

15. A kit for use in the detection of colorectal cancer, the kit comprising:
a swab sampling and sample transport system for the minimally invasive sampling of rectal mucosal cells, which system is comprised of:
a swab configured to sample colonic mucosal cells from the rectum; and
a collection container for receiving the swab after the sample has been taken, wherein the collection container is configured to stabilize, extract and store the sample; and
at least one reagent that is used in the analysis of polynucleotide expression levels, wherein the polynucleotides are selected from SEQ. ID Nos. 1-16.

16. A method for drug screening, the method comprising the following steps:
selecting a model biological system for at least one of colorectal cancer, lung cancer, prostate cancer, breast cancers, Alzheimer's and ALS;
selecting at least one prospective drug for screening using the suitable model biological system;
selecting at least two biomarkers from a panel of biomarkers identified by SEQ. ID 1-32;
dosing the model biological system with the at least one prospective drug; and
monitoring the response of the at least two biomarkers in the model biological system as a function of the dosing step.

17. The method of claim 16, further comprising: determining the efficacy of the prospective drug based on the monitoring step.
